(19) United States
(12) Patent Application Publication
(10) Pub. No.: US 2012/0309952 A1
(43) Pub. Date: Dec. 6, 2012

(54) SMALL RNA PURIFICATION

(75) Inventors: Rex M. Bitner, Cedarburg, WI (US); Mark A. Denhart, Madison, WI (US); Donald B. Smith, Evansville, WI (US)

(73) Assignee: PROMEGA CORPORATION, MADISON, WI (US)

(21) Appl. No.: 13/550,743

(22) Filed: Jul. 17, 2012

Related U.S. Application Data
(63) Continuation of application No. 11/715,761, filed on Mar. 8, 2007.
(60) Provisional application No. 60/780,089, filed on Mar. 8, 2006.

(51) Int. Cl. C07H 1/08; C07H 21/02; C12N 9/99

(52) U.S. Cl. 536/25.41; 435/184

(57) ABSTRACT

The present invention relates to methods, kits, and compositions for purifying small RNA molecules. In particular, the present invention provides methods for purifying small RNA molecules from a sample containing both small RNA molecules and larger RNA molecules using a compaction agent and a RNA binding matrix, as well as compositions and kits for practicing such methods. In certain embodiments, the compaction agent comprises a plurality of metal-amine-halide molecules.

### CROSS-REFERENCE TO RELATED APPLICATIONS

This application is a continuation of U.S. application Ser. No. 11/715,761 filed Mar. 8, 2007, which claims priority to U.S. Provisional Application No. 60/780,089 filed Mar. 8, 2006, the contents of which are both incorporated herein by reference.

The present invention relates to methods, kits, and compositions for purifying small RNA molecules. In particular, the present invention provides methods for purifying small RNA molecules from a sample containing both small RNA molecules and larger RNA molecules using a compaction agent and an RNA binding matrix, as well as compositions and kits for practicing such methods. In certain embodiments, the compaction agent comprises a plurality of metal-amine-halide molecules.

### BACKGROUND OF THE INVENTION

Interest in the identification, detection, and use of small RNAs has expanded rapidly in the last few years, particularly with the recent discoveries related to microRNAs and small interfering RNAs (siRNA), both of which have a powerful affect on the expression of genes. siRNA molecules, which are generally short, double stranded RNA, are used to silence the expression of specific genes at the post-transcriptional level by a pathway known as RNA interference (RNAi). microRNAs, small regulatory RNA molecules, have been shown to regulate target gene expression in various organisms. siRNA and microRNA molecules generally range between about 15 and 30 nucleotides in length. Other types of small RNAs include small nuclear RNAs (snRNAs) and small nucleolar RNAs (snoRNAs), both of which are involved in mRNA and rRNA processing, as well as tRNAs (about 70-90 bases), and 5S rRNA (about 120 bases), which are both involved in protein translation.

Historically, two basic methods have been used to isolate RNA molecules. The first is chemical extraction which usually employs concentrated chaotropic salts in combination with phenol or phenol-chloroform. This method is used to dissolve or precipitate proteins, allowing the protein-free phase to be separated by centrifugation. This type of method, while generally recovering very purified RNA, typically requires desalting and concentration with an alcohol precipitation step, which prevents the quantitative recovery of small RNA molecules.

The second method relies on selectively immobilizing RNA on a solid surface (generally glass) such that the proteins and debris can be washed away and the RNA eluted in an aqueous solution. This solid-phase type method relies on high salt or salt and alcohol to decrease the affinity of RNA for water and increase its affinity for the solid support used. The use of glass (silica) as a solid support has been shown to work for large RNAs, but is generally not considered useful for isolating small RNAs unless special procedures are employed involving both lysate purification as well as the use of two separate RNA binding and elution steps, as described in AMBION's mirVana™ miRNA Isolation Kit (see also, U.S. Pat Pub. 2005/0059024 to Conrade et al., herein incorporated by reference). The mirVana™ miRNA isolation procedure relies on a phenol-chloroform lysate purification step prior to RNA purification. This method also relies on the use of two silica binding membranes, with the first membrane used to bind large RNA molecules (with small RNA molecules flowing through the membrane) and the second membrane used to bind small RNA molecules.

What is needed, therefore, are methods and compositions that allow simple small RNA purification, without requiring the use of multiple binding membranes and/or without the need to purify the cell lysate prior to contacting with a binding membrane.

### SUMMARY OF THE INVENTION

The present invention relates to methods, kits, and compositions for purifying small RNA molecules. In particular, the present invention provides methods for purifying small RNA molecules from a sample containing both small RNA molecules and larger RNA molecules using a compaction agent and a RNA binding matrix, as well as compositions and kits for practicing such methods. In certain embodiments, the compaction agent comprises a plurality of metal-amine-halide molecules. In other embodiments, the compaction agent comprises a plurality of metal-amine-salt molecules (e.g. metal amide sulfate molecules).

In certain embodiments, the present invention provides methods for purifying small RNA molecules comprising: a) mixing a sample with a compaction agent, wherein the compaction agent comprises: i) a plurality of metal-amine-halide molecules, wherein the metal-amine-halide molecules comprise a metal atom, a halide atom, and at least one amine group (e.g. 2, 3, 4, 5 . . . 10 or 15 or more amine groups), and/or ii) or a plurality of metal-amine-salt molecules, wherein said metal-amine-salt molecules comprise a metal atom, a salt molecule, and at least one amine group; and wherein the sample comprises small RNA molecules and larger RNA molecules, and wherein the small RNA molecules are less than 1000 bases in length and the larger RNA molecules are longer than the small RNA molecules; and b) contacting the sample comprising the small and larger RNA molecules with a binding matrix such that a RNA-bound binding matrix is generated. In some embodiments, the present invention provides methods for purifying small RNA molecules comprising: a) mixing a sample with a compaction agent, wherein the compaction agent comprises: i) a plurality of metal-amine-halide molecules, wherein the metal-amine-halide molecules comprise a metal atom, a halide atom, and at least one amine group (e.g. 2, 3, 4, 5 . . . 10 or 15 or more amine groups), and/or ii) or a plurality of metal-amine-salt molecules, wherein said metal-amine-salt molecules comprise a metal atom, a salt molecule, and at least one amine group; and wherein the sample comprises small RNA molecules and larger RNA molecules, and wherein the small RNA molecules are less than 1000 bases in length and the larger RNA molecules are longer than the small RNA molecules; b) contacting the sample comprising the small and larger RNA molecules with a binding matrix such that a RNA-bound binding matrix is generated, and c) eluting small RNA molecules from the RNA-bound binding matrix such that a purified small RNA preparation is generated, wherein the purified small RNA preparation comprises a plurality of eluted small RNA molecules, and wherein the purified small RNA preparation is substantially free of larger RNA molecules. In certain embodiments, the methods further comprise washing the RNA-bound binding matrix of step (b) with a wash solution.

In particular embodiments, the halide atom is one of the following types of atoms: chlorine, fluorine, bromine, iodine, or astatine. In certain embodiments, the amount of the eluted small RNA molecules in the small RNA preparation is at least 5%, or at least 10% (e.g., 10%, 15%, 25%, 40%, 50%, 70%, 80% or 90%), of the small RNA molecules originally present in the sample prior to contacting with the binding matrix. In further embodiments, the purified small RNA preparation is essentially free of larger RNA molecules. In some embodiments, the purified small RNA preparation contains less than about 60, or 50, or 40 discreet larger RNA molecules. In particular embodiments, the contacting step of step (b) is conducted only once in order to generate the purified small RNA preparation.

In other embodiments, the sample in step a) further comprises DNA molecules, and wherein the purified small RNA preparation is substantially free of DNA molecules. In some embodiments, at least a portion of the DNA molecules are small DNA molecules less than about 100 base pairs in length, and wherein the purified small RNA preparation is substantially free of bound small DNA molecules.

In other embodiments, the methods further comprise mixing the sample with a salt solution. In particular embodiments, the concentration of salt in the sample prior to step (b), is between about 1.0 mM and about 400 mM. In some embodiments, the concentration of salt in the sample is below about 35 mM and the small RNA molecules are between 25 and 200 bases in length, or between 80-120 bases in length. In different embodiments, the concentration of salt in the sample is between 35 mM and 70 mM and the small RNA molecules are between 200 and 500, or between 300-400, bases in length. In further embodiments, the concentration of salt in the sample is between 70 mM and 400 mM and the small RNA molecules are between 500 and 1000 bases in length, or between 600-800 bases in length.

In some embodiments, the small RNA molecules are 950 bases in length or shorter. In certain embodiments, the small RNA molecules are 750 bases in length or shorter. In other embodiments, the small RNA molecules are 500 bases in length or shorter. In some embodiments, the small RNA molecules are 200 bases in length or shorter. In particular embodiments, the small RNA molecules are 100 bases in length or shorter. It is noted and intended that the present invention is not limited by the size of the small RNA molecules, as long as they are less than 1000 bases in length (e.g., less than or between 15 . . . 22 . . . 35 . . . 47 . . . 69 . . . 88 . . . 100 . . . 125 . . . 150 . . . 175 . . . 250 . . . 333 . . . 410 . . . 500 . . . 685 . . . 750 . . . 820 . . . 910 . . . 950 . . . or 999).

In other embodiments, the RNA-bound binding matrix produced in step (b) is washed with a wash solution. In certain embodiments, the wash solution contains an alcohol. In further embodiments, the alcohol is selected from the group consisting of ethanol, methanol, isopropanol and propanol. In additional embodiments, the wash solution comprises ethanol. In certain embodiments, the ethanol is present in the solution at a concentration of between about 20-40 percent.

The present invention is not limited by the type of compaction agent and instead contemplates any compaction agent that is configured to (1) allow a RNA binding matrix to preferentially bind small RNA molecules over larger RNA molecules and/or (2) preferentially elute small RNA molecules over larger RNA molecules from the RNA-bound matrix. In certain embodiments, the compaction agent includes, but is not limited to: a basic polypeptide, polylysine, a polyamine, protamine, spermidine, spermine, putrescine, cadaverine, a trivalent metal ion, a tetravalent metal ion, hexammine cobalt chloride, chloropentammine cobalt, chromium, netropsin, monomethylamminepentaammine cobalt chloride, distamycin, lexitropans, hexamethylammine cobalt chloride, DAPI (4′,6 diamino 2-phenylindol), berenil, pentamidine, and manganese chloride. In other embodiments, the compaction agent comprises cobalt. In further embodiments, the compaction agent comprises hexammine cobalt chloride. In particular embodiments, the compaction agent comprises a plurality of metal-amine-halide molecules. In some embodiments, the compaction agent is selected from the group consisting of: nickel hexammine chloride, ruthenium hexammine chloride, hexammine cobalt chloride, and chloropentammine cobalt chloride. In other embodiments the compaction agent is selected from the group consisting of: cobalt hexaethanolamine chloride, cobalt monoethanolamine pentaethylamine chloride, cobalt diethanolamine tetraethylamine chloride, cobalt triethanolamine triethylamine chloride, cobalt tetraethanolamine diethylammine chloride, cobalt pentaethanolamine monoethylamine chloride, cobalt hexaethylamine chloride, cobalt hexaethanolamine sulfate, cobalt monoethanolamine pentaethylamine sulfate, cobalt diethanolamine tetraethylamine sulfate, cobalt triethanolamine triethylamine sulfate, cobalt tetraethanolamine diethylammine sulfate, cobalt pentaethanolamine monoethylamine sulfate, cobalt hexaethylamine sulfate, or mixtures thereof. In other embodiments the compaction agent is selected from the group consisting of: nickel hexaethanolamine chloride, nickel monoethanolamine pentaethylamine chloride, nickel diethanolamine tetraethylamine chloride, nickel triethanolamine triethylamine chloride, nickel tetraethanolamine diethylammine chloride, nickel pentaethanolamine monoethylamine chloride, nickel hexaethylamine chloride, nickel hexaethanolamine sulfate, nickel monoethanolamine pentaethylamine sulfate, nickel diethanolamine tetraethylamine sulfate, nickel triethanolamine triethylamine sulfate, nickel tetraethanolamine diethylammine sulfate, nickel pentaethanolamine monoethylamine sulfate, nickel hexaethylamine sulfate, or mixtures thereof.

In certain embodiments, the concentration of compaction agent in the sample prior to step (b), is between about 2.0 mM and about 8.0 mM (e.g., about 2.0 mM, about 4.0 mM, about 6.0 mM, or about 8.0 mM; although the present invention is not limited to these concentration ranges). In certain embodiments, the composition further comprises a buffer. In some embodiments, the buffer is selected from the group consisting of: HEPES, MES, and TRIS. In particular embodiments, the buffer has a pH between about 5.5 and about 9.0 (e.g. 5.5, 6.5, 7.5, 8.5 or 9.0).

In some embodiments, the methods further comprise contacting the sample with a chaotropic agent, wherein the chaotropic agent comprises an amide. In other embodiments, the chaotropic agent is selected from urea, thiourea, and acetamide. In particular embodiments, the methods further comprise contacting the sample with a chaotropic agent, wherein the chaotropic agent comprises a urethane group. In further embodiments, the chaotropic agent comprises urethane. In other embodiments, the methods further comprise contacting the sample with a chaotropic agent, wherein the chaotropic agent comprises urea-like molecules. In particular embodiments, the sample does not contain a chaotropic agent during step (b).

In certain embodiments, the sample comprises a cell lysate, wherein the cell lysate comprises lysed cells. In some embodiments, the sample contains the cell lysate during the contacting step (e.g., the cell lysate is not purified away from the sample prior to contact with the binding matrix). In certain embodiments, the binding matrix is a membrane (e.g. silica membrane, cellulose acetate membrane, nylon membrane). In particular embodiments, the binding matrix comprises a solid support. In some embodiments, the binding matrix comprises silica. In further embodiments, the binding matrix comprises magnetic particles. In some embodiments, the binding matrix comprises silica and Fe₃O₄, or silica and Fe₂O₃.

In certain embodiments, the cell lysate is generated from cells (e.g. human, murine, E. coli, etc.) susceptible to lysis using chaotropic agents such as urea, thiourea, acetamide and urethane. In other embodiments, the cell lysate is generated from cells that may require a pre-treatment step because of special cell wall structures, such as plant cells, yeast cells, fungus cells, and certain gram positive bacteria cells. These types of cells may be pretreated (e.g. protoplasted) and then processed by the methods and compositions of the present invention.

In certain embodiments, the purified small RNA preparation is enriched for small RNA molecules compared to the original sample. For example, small RNA in a sample may be enriched (e.g., as measured by UV absorption) about or at least about 2-fold, 3.5-fold, 5-fold, 10-fold, 50-fold, 100-fold, 150-fold, 200-fold, 500-fold, 800-fold, 1000-fold, 2000-fold, and all ranges therein as determined by the concentration (e.g. ug/ml) or mass of small RNA molecules relative to the concentration or mass of total RNA molecules prior to contacting the original sample with the binding matrix compared to after eluting the small RNA molecules from the binding matrix. Enrichment and/or purification may also be measured in terms of the number of small RNA molecules relative to the number of total RNA molecules present in the original sample. Small RNA molecules can be isolated such that a sample is enriched (e.g., as measured by UV absorption) about or at least about 2-fold, 3.5-fold, 5-fold, 10-fold, 50-fold, 100-fold, 150-fold, 200-fold, 500-fold, 800-fold, 1000-fold, 2000-fold, and all ranges therein in small RNA molecules as determined by number of small RNA molecules relative to total number of RNA molecules prior to contacting the original sample with the binding matrix compared to after eluting the small RNA molecules from the binding matrix. Enrichment and/or purification of small RNAs may also be measured in terms of the increase of small RNA molecules relative to the number of total RNA molecules. Small RNA molecules can be isolated such that the amount of small RNA molecules is increased about or at least about 10%, 15%, 20%, 25%, 30%, 35%, 40%, 45%, 50%, 55%, 60%, 65%, 70%, 75%, 80%, 85%, 90%, 95% or more with respect to the total amount of RNA in the sample before and after isolation. In certain embodiments, the enrichment and/or purification of small RNA molecules can be quantified in terms of the absence of larger RNA molecules present in the sample after eluting the RNA from binding matrix. Small RNA molecules can be enriched such that the number of larger RNA molecules by mass in the small RNA preparation after eluting the RNA from the binding matrix is no more than about 30%, 25%, 20%, 15%, 10%, 5%, 4%, 3%, 2%, 1%, 0.5%, 0%, or any range therein of the RNA eluted from the binding matrix. In some embodiments, at least about 10%, 15%, 20%, 25%, 30%, 35%, 40%, 45%, 50%, 55%, 60%, 65%, 70%, 75%, 80%, 85%, 90%, 95% of the small RNA molecules in the original sample are isolated after small RNA molecules are eluted from the binding matrix.

In some embodiments, the amount of eluted small RNA molecules in the small RNA preparation is at least 5% of the small RNA molecules originally present in the sample prior to contacting with the binding matrix. In other embodiments, the amount of eluted small RNA molecules in the small RNA preparation is at least 15% of the small RNA molecules originally present in the sample prior to contacting with the binding matrix (e.g. at least 15% . . . 25% . . . 40% . . . 50% . . . 65% or at least 75%). In further embodiments, the amount of eluted small RNA molecules in the small RNA preparation is between 5-50% of the small RNA molecules originally present in the sample prior to contacting with the binding matrix (e.g. between 10-30% or between 15-20%).

In certain embodiments, the methods further comprise the step of using or characterizing the small RNA molecules in the purified small RNA preparation. After RNA is eluted individual or specific small RNA molecules and/or preparations of small RNA molecules (as well as the entire population of isolated small RNA molecules) can be subject to additional reactions and/or assays. In some cases, these reactions and/or assays involve amplification of the small RNA molecules. For example, RT-PCR may be employed to generate molecules that can be characterized. In some embodiments, a particular small RNA molecule or a small RNA preparation may be quantified or characterized. Quantification includes any procedure known to those of skill in the art such as those involving one or more amplification reactions or nuclease protection assays, such as those using ribonuclease to discriminate between probe that is hybridized to a specific miRNA target or unhybridized, as embodied in the mirVana miRNA Detection Kit from Ambion. These procedures also include quantitative reverse transcriptase-PCR (qRT-PCR, such as Applied Biosystem's TaqMan Micro RNA assays). In some embodiments, characterization of the isolated small RNA is performed. Other characterization and quantification assays are contemplated as part of the invention. The small RNA molecules can also be used with arrays; to generate cDNAs for use in arrays or as targets to be detected by arrays, or after being labeled by radioactive, fluorescent, or luminescent tags. Other assays include the use of spectrophotometry, electrophoresis, and sequencing. In certain embodiments, the small RNA molecules are used for research, diagnostics, or therapy.

In particular embodiments, a chaotropic agent is employed comprising urea. In certain embodiments, the chaotropic agent contains free urea molecules. In other embodiments, the chaotropic agent comprises urea-containing compounds.

In some embodiments, the present invention provides kits for purifying small RNA molecules, comprising; a) a vessel containing a compaction agent, wherein the compaction agent comprises a plurality of metal-amine-halide molecules or metal amine salt molecules (e.g. metal-amine-sulfate), wherein the metal-amine-halide molecules comprise a metal atom, a halide atom, and at least one amine group and the metal amine salt molecules comprise a metal atom, a salt molecule, and at least one amine group; and b) a binding matrix, wherein the binding matrix is configured to bind RNA molecules.

In certain embodiments, the kits further comprise a chaotropic agent, wherein the chaotropic agent comprises an amide. In other embodiments, the chaotropic agent is selected from the group consisting of: urea, thiourea, and acetamide. In some embodiments, the kits further comprise a chaotropic agent, wherein the chaotropic agent comprises a urethane group.

In other embodiments, the kits further comprise a binding column. In some embodiments, the kit further comprises a written insert component that comprises instructions for using the compaction agent to purify small RNA molecules from a sample comprising small RNA molecules and larger RNA molecules, wherein the small RNA molecules are less than 1000 bases in length and the larger RNA molecules are longer than the small RNA molecules.

In particular embodiments, the present invention provides compositions comprising a chaotropic agent selected from urea, thiourea, acetamide, and urethane and a compaction agent, wherein said compaction agent comprises: i) a plurality of metal-amine-halide molecules, wherein the metal-amine-halide molecules comprise a metal atom, a halide atom, and a plurality of amine groups, and/or ii) or a plurality of metal-amine-salt molecules, wherein said metal-amine-salt molecules comprise a metal atom, a salt molecule, and at least one amine group. In certain embodiments, the compositions further comprise a buffer. In other embodiments, the buffer is selected from the group consisting of: HEPES, MES, and TRIS. In some embodiments, the buffer has a pH between about 5.5 and about 9.0. In certain embodiments, the compaction agent comprises hexamine cobalt chloride. In other embodiments, the compositions further comprise a sample comprising small RNA molecules and larger RNA molecules, wherein the small RNA molecules are less than 1000 bases in length and the larger RNA molecules are longer than the small RNA molecules.

In some embodiments, the present invention provides a system comprising a container, a binding matrix and a purified small RNA preparation, wherein said binding matrix and the purified small RNA preparation are located within the container, wherein the binding matrix comprises bound larger RNA molecules, and wherein the purified small RNA preparation comprises a plurality of small RNA molecules and is substantially free of larger RNA molecules, and wherein the small RNA molecules are less than 1000 bases in length and the larger RNA molecules are longer than the small RNA molecules.

In certain embodiments, the container comprises a plate with a plurality of wells. In other embodiments, at least a portion of the wells of the plate have bottom portions adapted to be mounted to a vacuum system. In other embodiments, the wells of the plate are fully enclosed (e.g., not configured to be attached to a vacuum system). In particular embodiments, the container comprises a tube or column.

In certain embodiments, the present invention provides purified small RNA preparations comprising a plurality of small RNA molecules and a compaction agent, wherein the compaction agent comprises: i) a plurality of metal-amine-halide molecules, wherein the metal-amine-halide molecules comprise a metal atom, a halide atom, at least one amine group, and/or ii) or a plurality of metal-amine-salt molecules, wherein said metal-amine-salt molecules comprise a metal atom, a salt molecule, and at least one amine group, wherein the purified small RNA preparation is substantially free of larger RNA molecules, and wherein the small RNA molecules are less than 1000 bases in length and the larger RNA molecules are longer than the small RNA molecules.

In certain embodiments, the present invention provides methods of reducing the degradation of RNA in a sample by RNase comprising contacting a RNA-containing sample with a compound selected from the group consisting of a chaotropic agent, a compaction agent and mixtures thereof. In some embodiments, the chaotropic agent is selected from the group consisting of urea, urethane and acetamide. In particular embodiments, the sample is a cell lysate.

In some embodiments, the present invention provides a modified binding matrix comprising: a) a compaction agent comprising: i) a plurality of metal-amine-halide molecules, wherein the metal-amine-halide molecules comprise a metal atom, a halide atom, and at least one amine group, and/or ii) a plurality of metal-amine-salt molecules, wherein the metal-amine-salt molecules comprise a metal atom, a salt molecule, and at least one amine group; and b) a binding matrix, wherein at least a portion of the binding matrix is impregnated with, coated with, or impregnated and coated with the compaction agent. In certain embodiments, the modified binding matrix is configured to purify small RNA molecules from a sample.

In particular embodiments, the present invention provides methods for purifying small RNA molecules comprising: a) providing a modified binding matrix comprising; i) a compaction agent comprising: A) a plurality of metal-amine-halide molecules, wherein the metal-amine-halide molecules comprise a metal atom, a halide atom, and at least one amine group, and/or B) a plurality of metal-amine-salt molecules, wherein the metal-amine-salt molecules comprise a metal atom, a salt molecule, and at least one amine group; and ii) a binding matrix, wherein at least a portion of the binding matrix is impregnated with, coated with, or impregnated and coated with the compaction agent; b) contacting a sample with the modified binding matrix, wherein the sample comprises small RNA molecules and larger RNA molecules, and wherein the small RNA molecules are less than 1000 bases in length and the larger RNA molecules are longer than the small RNA molecules, such that an RNA-bound binding matrix is generated, and c) eluting small RNA molecules from said RNA-bound binding matrix such that a purified small RNA preparation is generated, wherein the purified small RNA preparation comprises a plurality of eluted small RNA molecules, and wherein the purified small RNA preparation is substantially free of larger RNA molecules.

In certain embodiments large RNA molecules are bound to the matrix, and small RNA are less than 1000 bases in length and are not substantially bound to the matrix. In particular embodiments, the small RNA preparation is substantially free of large RNA molecules of more than 1000 bases in length.

In certain embodiments, the present invention provides methods for purifying small RNA molecules comprising: contacting a sample with a binding matrix wherein the binding matrix comprises a compaction agent bound to the binding matrix surface, for example by depositing the compaction agent onto the binding matrix surface prior to contact of the binding matrix with the sample, by means such as precipitation of the compaction agent on the binding matrix surface or by passing a solution containing compaction agent under such conditions that result in the compaction agent being deposited onto the binding matrix surface.

### DESCRIPTION OF THE FIGURES

FIG. 1 shows the results from Example 1, which describes the purification of RNA from a cell lysate using a compaction agent and different ratios of GITC (guanidinium isothiocyanate) and urea without using a separate lysate purification step. FIG. 1A shows the RNA from samples that were in 0% ethanol when passed through the SV mini column;

FIG. 1B shows the RNA from the samples that were in 25% ethanol when passed through the SV mini column; and FIG. 1C shows the RNA from the samples that were in 50% ethanol when passed through the SV mini column.

FIG. 2 shows the results from Example 2, which describes the small RNA purification from a cell lysate using urea, a compaction agent, and various concentrations of NaCl.

FIG. 3 shows the results from Example 3, which describes the small RNA purification from a cell lysate using urea and various concentrations of compaction agent Hexamminecobalt(III)chloride.

FIG. 4 shows the results from Example 4, which describes the small RNA purification from yeast cells.

FIG. 5 shows the results from Example 5, which describes the small RNA purification from a cell lysate of E. coli cells using just a single binding column membrane without using a separate lysate purification step.

FIG. 6 shows the results from Example 6, which describes the small RNA purification from a human cell lysate using urea, a compaction agent, and various buffers at various pHs.

FIG. 7 shows the results from Example 7, which describes the small RNA purification from a human cell lysate using a compaction agent and various chaotropic agents, including urea, thiourea, acetamide and urethane.

FIG. 8 shows the results from Example 8, which describes the small RNA purification from beef tissue using a compaction agent and various chaotropic agents, including urea, thiourea, acetamide, and urethane.

FIG. 9 shows the results from Example 9, which describes the small RNA purification from human cells using urea, a compaction agent, and isopropanol.

FIG. 10 shows the results from Example 10, which describes the small RNA purification from human cells using urea, a compaction agent, and methanol.

FIG. 11 shows the results from Example 11, which describes the small RNA purification from plant tissue using a compaction agent and various chaotropic agents.

FIG. 12 shows the results from Example 12, which describes the purification of small RNA from a mixture of RNA using acetamide and either no alcohol or various concentrations of alcohol. The results in FIG. 12A are the result of using a SV membrane and the results in FIG. 12B the result of using a nylon membrane.

FIG. 13 shows the results from Example 13, which describes the purification of small RNA from a mixture of RNA using acetamide and either no alcohol or various concentrations of alcohol and various membranes. FIG. 12A shows the results using a SV membrane, FIG. 13B shows the results using a nylon membrane, and FIG. 13C shows the results using a cellulose acetate membrane.

FIG. 14 shows the results of Example 14, which describes the purification of small RNA from a mixture of RNA using no chaotrope and either no alcohol or various concentrations of alcohol. FIG. 14A shows the results using a nylon membrane, while FIG. 14B shows the results using a cellulose acetate membrane.

FIG. 15 shows the results of Example 15, which describes the purification of small RNA using either no wash step or a wash step with different ethanol concentrations. FIG. 15A shows the results using a nylon membrane, FIG. 15B shows the results with a cellulose acetate membrane, and FIG. 15C shows the results using a SV membrane.

FIG. 16 shows the results of Example 16, which describes small RNA purification using silica-magnetic particles and various chaotropes.

FIG. 17 shows the results of Example 17, which describes the purification of small RNA using SV96 binding plates and various chaotropes.

FIG. 18 shows the results of Example 18, which describes the purification of small RNA using hexamminenickel chloride and acetamide.

FIG. 19 shows the results of Example 19, which describes the purification of small RNA using hexammine nickel chloride and various percentage ethanol rinses.

FIG. 20 shows the results of Example 20, which describes the purification of small RNA using ruthenium hexammine trichloride and acetamide.

FIGS. 21A and B show the results from Example 25, which describes the binding and elution of RNA bound to columns pretreated with hexammine cobalt chloride

FIGS. 22A and B show the results from Example 29, which describes methods of screening the binding of transition metal complexes to a mixture of oligonucleotides.

### DEFINITIONS

To facilitate an understanding of the invention, a number of terms are defined below.

As used herein, the phrase “a sample that comprises small RNA molecules and larger RNA molecules,” when used in reference to small RNA molecules being less than 1000 bases in length and larger RNA molecules that are longer than the small RNA molecules, refers to any type of sample, such as biological or environmental samples, that includes a detectable quantity of both small RNA molecules and larger RNA molecules for a given size of small RNA molecules (e.g. 500 bases). For example, a sample that contains RNA molecules that are 400 bases and 600 bases in length, where the 400 base sequences bind to the binding matrix after being processed according to the present invention and those that are 600 bases do not substantially bind to the binding matrix, is an exemplary sample since it contains both small RNA molecules (the 400 base sequences that will bind to the binding matrix) and larger RNA molecules (the 600 base sequences that do not substantially bind). Specific examples of sources of such samples, as long as they have these two species of RNA molecules, include: a cell lysate, a previously purified RNA sample, an RNA control sample, a pharmaceutical drug preparation, a protein preparation, a lipid preparation, as well as animal fluid samples such as blood, plasma, serum, or semen.

As used herein, the phrase “binding matrix” refers to any type of substrate, whether porous or non-porous, that will bind RNA molecules in the presence of a compaction agent such that small RNA molecules can be preferentially eluted therefrom to generate purified small RNA preparations that are substantially free of larger RNA molecules. Examples of such binding matrices include, but are not limited to, nylon membranes or particles, silica membranes or particles, cellulose acetate membranes or particles, membranes or particles composed of silica and Fe₃O₄, and other similar membranes, fibers, coated plates, solid supports, and particles. The ability of a particular material to serve as a binding matrix in the methods of the present invention can be determined, for example, by substituting in the candidate material in Example 1-30 below and reviewing the resulting gel to determine if the candidate material is able to serve as a binding matrix.

As used herein, a purified small RNA sample or purified small RNA preparation is considered “substantially free of larger RNA molecules” when, of all the RNA present in the sample, less than 5.0% of the total RNA is larger RNA (i.e. at least 95.1% of the total RNA present is small RNA). The amount of RNA present may be determined by UV absorption methods or other methods used to quantitate RNA molecules.

As used herein, a purified small RNA sample or purified small RNA preparation is considered “essentially free of larger RNA molecules” when, of all the RNA present in the sample, less than 1.0% of the total RNA is larger RNA (i.e. at least 99.1% of the total RNA present is small RNA). The amount of RNA present may be determined by UV absorption methods or other methods used to quantitate RNA molecules.

As used herein, the term “amine group” refers to structures of the formula:

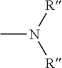

where R″ is independently hydrogen or R′, and R′ is substituted or unsubstituted alkyl, alkenyl, cycloalkyl, cycloalkenyl and aryl.

As used herein, the term “ammine” refers to a species of amine comprising the coordination of a metal atom with a plurality of ammonium groups. At least one of the hydrogens in at least one of the ammonium groups may be substituted with alkyl, alkenyl, cycloalkyl, cycloalkenyl and aryl.

As used herein, the phrase “metal-amine-halide molecule” refers to any molecule that contains a metal atom, a halide atom, and at least one amine group. Examples of such molecules include, but are not limited to: hexammine cobalt chloride, monomethylamminepentammine cobalt chloride, monoethylamminepentammine cobalt chloride, dimethylamminetetraammine cobalt chloride, trimethylamminetriammine cobalt chloride, hexamethylammine cobalt chloride, hexaethylammine cobalt chloride, hexammine nickel chloride, monomethylamminepentammine nickel chloride, trimethylamminetriammine nickel chloride, ruthenium hexammine trichloride, ruthenium dimethylamminetetraammine trichloride, and similarly substituted compounds in which iridium is the coordinated metal atom. In the literature, some of these compounds are sometimes referred to using the term “amine”, rather than “ammine”, as exemplified in “hexamine cobalt chloride”.

As used herein, the phrase “metal amine salt molecules” refers to any molecule that contains a metal atom, a salt molecule, and at least one amine group. Examples of such molecules include, but are not limited to, cobalt hexaethanolamine chloride, cobalt monoethanolamine pentaethylamine chloride, cobalt diethanolamine tetraethylamine chloride, cobalt triethanolamine triethylamine chloride, cobalt tetraethanolamine diethylammine chloride, cobalt pentaethanolamine monoethylamine chloride, cobalt hexaethylamine chloride, cobalt hexaethanolamine sulfate, cobalt monoethanolamine pentaethylamine sulfate, cobalt diethanolamine tetraethylamine sulfate, cobalt triethanolamine triethylamine sulfate, cobalt tetraethanolamine diethylammine sulfate, cobalt pentaethanolamine monoethylamine sulfate, cobalt hexaethylamine sulfate, nickel hexaethanolamine chloride, nickel monoethanolamine pentaethylamine chloride, nickel diethanolamine tetraethylamine chloride, nickel triethanolamine triethylamine chloride, nickel tetraethanolamine diethylammine chloride, nickel pentaethanolamine monoethylamine chloride, nickel hexaethylamine chloride, nickel hexaethanolamine sulfate, nickel monoethanolamine pentaethylamine sulfate, nickel diethanolamine tetraethylamine sulfate, nickel triethanolamine triethylamine sulfate, nickel tetraethanolamine diethylammine sulfate, nickel pentaethanolamine monoethylamine sulfate, and nickel hexaethylamine sulfate.

### DESCRIPTION OF THE INVENTION

The present invention relates to methods, kits, and compositions for purifying small RNA molecules. In particular, the present invention provides methods for purifying small RNA molecules from a sample containing both small RNA molecules and larger RNA molecules using a compaction agent and a RNA binding matrix, as well as compositions and kits for practicing such methods. In certain embodiments, the compaction agent comprises a plurality of metal-amine-halide molecules.

The compositions and methods of the present invention allow small RNA molecules to be purified from samples containing both small and larger RNA molecules. As shown in the Examples, the methods of the present invention allow such samples to be contacted with a binding matrix, such as a silica membrane, and a compaction agent such that a purified small RNA preparation is generated (that is substantially free or larger RNA molecules) when RNA is eluted from the binding matrix. Generating such a purified small RNA preparation by preferentially eluting small versus larger RNA molecules is unexpected as procedures in the art utilizing binding membranes and elution lead to the generation of RNA samples containing larger RNA molecules. Indeed, the art has proposed extensive procedures to deal with larger RNA molecule preferential purification involving both lysate purification as well as the use of two separate RNA binding and elution steps (See, AMBION's mirVana™ miRNA Isolation Kit, and U.S. Pat Pub. 2005/0059024 to Conrade et al., herein incorporated by reference). Surprisingly, the present invention allows one to not only avoid the need for two or more separate RNA binding and elution steps, but also removes the requirement for purifying the lysate prior to contact with the binding matrix (e.g. silica membrane). The present invention, which avoids the need for time consuming and extensive processing of samples, therefore satisfies the need in the art for simple and efficient methods for purifying small RNA molecules. Examples of procedures in the art that benefit from purified small RNAs, include microRNAs and small interfering RNAs (siRNA) based technologies, or other procedures that benefit from purified small RNAs.

### I. Small RNA Purification

Small RNA molecules may be purified from samples containing both small and larger RNA molecules using the methods compositions of the present invention. For example, in preferred embodiments, a compaction agent comprising a plurality of metal-amine-halide molecules is added to a sample which is then contacted with a binding matrix which will bind RNA. The small RNA molecules may then be preferentially eluted from the binding matrix to generate purified small RNA samples that are substantially free or larger RNA molecules. In certain embodiments, chaotropic agents such as urea, thiourea, acetamide, and urethane are employed.

Small RNA molecules may be isolated and purified according to the present invention from any type of nucleic acid preparation, biological sample, cell lysate, tissue homogenate, or any other type of sample that contains both the desired small RNA and larger, non-desired RNA molecules. Exemplary samples include, but are not limited to, blood, urine, endocrine fluid, tissues, cells, and lysates of tissues or cells. In certain preferred embodiments, the sample comprises a cell lysate.

Cell lysates may be prepared, for example, by methods known in the art. Generally, a cell suspension, tissue, organ, plant leaves, or other source of cells is mixed with a lysis buffer comprising a chaotropic salt in order to rupture the cells. The mixture is rapidly homogenized, using, for example, a hand held homogenizer or an automatic homogenizer, such as a Waring blender, a Polytron tissue homogenizer, or the like. After the cells are lysed (if the original sample contains cells) the sample containing both small RNA and larger RNA molecules is contacted with a compaction agent, and in some embodiments, a chaotrope such as urea, thiourea, acetamide or urethane.

In regard to the compaction agent, it is not intended that the present invention be limited to any particular compaction agent. In certain embodiments, the compaction agent is selected from the group consisting of: a basic polypeptide, polylysine, a polyamine, protamine, spermidine, spermine, putrescine, cadaverine, a trivalent metal ion, a tetravalent metal ion, hexammine cobalt, chloropentammine cobalt, chromium, netropsin, distamycin, lexitropans, DAPI (4′,6 diamino 2-phenylindol), berenil, pentamidine, and manganese chloride. In particular embodiments, the compaction agent comprises a plurality of metal-amine-halide molecules. In some embodiments, the compaction agent is selected from the group consisting of: nickel hexamine chloride, ruthenium hexamine chloride, hexamine cobalt chloride, and chloropentammine cobalt chloride.

In order to determine if a particular compaction agent may serve as a useful compaction agent in an embodiment of the present invention, one can screen such compounds using, for example, the same procedures as described in Examples 1-30, by replacing the compaction agent used in these examples with a candidate compaction agent to determine the degree to which the candidate compaction agent functions to permit selection of small RNAs. For example, one can determine the degree to which the compound causes smaller RNA molecules, rather than larger RNA molecules to be preferentially eluted from a binding matrix following the protocols in Examples 1-30.

As noted above, in certain embodiments, the sample is also contacted with a chaotropic agent such as urea, thiourea, acetamide, or other amides, or a chaotropic agent such as urethane or a compound containing urethane groups. With regard to the chaotropic agents comprising urea, such compositions may contain, for example, free urea molecules or molecules containing urea as a substituent. In certain embodiments, a composition is employed that contains urea-like, urea related, or urea containing molecules. Examples of such compounds include, for example, urea, 1,1-diethyl urea, 1,3-dimethyl urea and, n-methyl urea, thiourea, and urethane. Additional urea-like or urea-related compounds may be found in U.S. Pat. No. 6,670,332, and McElroy et al., J. Med. Chem. 46(6), 1066-1080, 2003 (which discusses 348 urea-like compounds). Chaotropic agents suitable for use in the present invention may be screened using, for example, the same procedures as Examples 1-30, by replacing the chaotropic agent described in the particular example with a candidate chaotropic agent. For example, one can determine the degree to which the compound, when combined with a compaction agent, causes smaller RNA molecules to be preferentially eluted from a binding matrix following the protocols in Examples 1-30.

In certain embodiments, an alcohol solution is also added to the sample. In particular embodiments, the alcohol is added to the sample at a concentration of about 15-35% (e.g. 25% ethanol). The alcohol solution can be about, be at least about, or be at most about 5, 10, 15, 20, 25, 30, 35, 40, 45, 50, 55, 60, 65, 70, 75, 80, 85, 90, or 99% alcohol, or any range therein. In certain embodiments, the alcohol is added to the sample to make the sample have a concentration of alcohol of about, about at least, or about at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90%, or any range therein. In specific embodiments, the amount of alcohol added to a lysate renders it with an, alcohol concentration of about 15% to about 50%. In specific embodiments, the amount of alcohol solution added to the sample gives it an alcohol concentration of about 25%. Alcohols include, but are not limited to, ethanol, propanol, isopropanol, and methanol.

In certain embodiments, the binding matrix comprises a binding column (e.g. with a silica membrane). A description of such binding columns is provided in U.S. Pat. No. 6,218,531, herein incorporated by reference in its entirety. In certain embodiments, the RNA bound binding matrix is washed with a wash solution to remove salts and other debris.

The small RNA can be eluted from the RNA bound binding matrix using standard methods. For example, nuclease free water may be employed to elute the bound RNA molecules such that a purified small RNA preparation is generated.

### II. Quantifying Small RNA

Small RNAs may be quantitated by any method to determine, for example, the amount or concentration of small RNA molecules that are present. Preferably, the small RNAs are quantitated to determine that amount or concentration that is bound to a RNA binding matrix (e.g. after the contacting step), or the amount that is eluted into a purified small RNA preparation (e.g. to determine how much of the RNA in the purified small RNA preparation is small versus larger RNA molecules, or to determine what percent of small RNA molecules from the original sample are present in the purified small RNA preparation). Exemplary quantitation methods are provided in U.S. Pat. Pub. 2005/0059024 (herein incorporated by reference in its entirety) and as discussed below.

RNA may be quantitated using UV absorbance. For example, the concentration and purity of RNA can be determined by diluting an aliquot of the preparation (e.g., a 1:50 to 1:100 dilution) in TE (10 mM Tris-HCl pH 8, 1 mM EDTA) or water, and reading the absorbence in a spectrophotometer at 260 nm and 280 nm. An A₂₆₀ of 1 is equivalent to about 40 ug RNA/ml. The concentration (ug/ml) of RNA may therefore be calculated by multiplying the A₂₆₀ X dilution factor X 40 ug/ml.

Small RNA molecules isolated from a sample may be also quantitated by gel electrophoresis using a denaturing gel system. Acrylamide gels are suitable gels for separations of this size, although high concentrations (about 4%) of modified agarose can also be used. A positive control should generally be included on the gel so that any unusual results can be attributed to a problem with the gel or a problem with the RNA under analysis. RNA molecular weight markers, a RNA sample known to be intact, or both, can be used for this purpose. It is also a good idea to include a sample of the starting RNA that was used in the enrichment procedure. The amount of small RNA molecules present in any given band can be quantitated by, for example, comparison to control bands on the same gel.

Additional quantitative methods include quantitative RT-PCR methods, in which the prevalence of certain RNA sequences can be compared within a RNA sample and between different RNA samples. Further the comparison of peak heights generated using systems such as the Agilent Bioanalyzer may also be used with internal standards to quantify and compare certain RNA sizes.

III. Purification of Small Interfering RNAs (siRNA) and micro RNA (miRNAs)

In certain preferred embodiments, the methods and compositions of the present invention are used to purify small interfering RNA molecules (siRNA) molecules and micro RNAs (miRNAs). Preferably, the siRNA and miRNA molecules are purified such that they may be used to perform or study RNA interference (RNAi) and related pathways.

RNAi represents an evolutionary conserved cellular defense for controlling the expression of foreign genes in most eukaryotes, including humans. RNAi is triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Purified siRNAs molecules have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference). The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference. siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing Brummelkamp et al, Science 2002; 296:550-3; and Holen et al, Nucleic Acids Res. 2002; 30:1757-66, both of which are herein incorporated by reference.

Micro RNAs (miRNAs) are small cellular RNAs that bind to the 3′UTR, and in mammalian cells are thought to inhibit translation of a targeted message (some may mediate cleavage). They generally contain at least one mismatch to their target sequence. This is in contrast to siRNAs, which are thought to promote cleavage of mRNAs and generally do not contain mismatches to their target sequence. It appears that miRNAs may very well regulate expression of a wide variety of genes—not just genes involved in developmental and neuronal cells, although an understanding of the mechanism is not necessary to practice the present invention and the present invention is not limited to any particular mechanism. miRNAs are expressed in the cell as 100-500 bp precursor RNAs (pre-miRNA), which are processed to form ˜70 bp pri-miRNAs, which are processed to form mature ˜17-22 base miRNAs. To understand the regulation of genes by miRNAs researchers express either the long pre-miRNA or the mature miRNA.

As such, in preferred embodiments, the methods and compositions of the present invention are employed to purify siRNA or miRNA molecules. For example, the methods are performed such that small RNAs less than about 200 bases or less than 100 bases are purified from larger RNAs (e.g., by altering the concentration of salt in the sample). In certain embodiments, the original sample contains cells that have been transformed with vectors expressing desired siRNA or miRNA molecules.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); ×g (times gravity); mg (milligrams); μg (micrograms); ng (nanograms); 1 or L (liters); ml (milliliters); μl (microliters); and C (degrees Centigrade).

### Example 1

### RNA Purification with a Compaction Agent and Different Ratios of GITC and Urea

This example describes the purification of RNA from a cell lysate using a compaction agent and different ratios of GITC and urea without using a separate lysate purification step. Five tubes, each containing 1×10⁶ cultured 293T human cells were centrifuged at 8,000×g and rinsed twice with 500 μl 1×PBS (phosphate buffered saline) pH 6.8 to remove cell culture media. PBS supernatant was removed after centrifugation of cells. To each of five tubes (tubes A-E) containing washed cells 4 M GITC (guanidine thiocyanate), 10 mM TRIS (tris(hydroxymethyl)aminomethane hydrochloride) pH 7.5 and/or 8 M Urea, 20 mM TRIS pH 7.5 was added in the following ratios: Tube A 175 μl GITC+0 μl Urea, Tube B. 130 μl GITC+45 μl Urea, Tube C. 85 μl GITC+90 μl Urea, Tube D. 45 μl GITC+130 μl Urea, Tube E. 0 μl GITC+175 μl Urea. Tubes were vortexed to resuspend cells. To each tube was added: (1) 2.5 μl 5 M NaCl in water and (2) 20 μl 250 mM Hexamminecobalt(III)chloride (Sigma Aldrich, St. Louis, Mo.) in 1×TE pH 8.0 (10 mM TRIS, 1 mM EDTA (ethylenediaminetetraacetic acid).

Tubes were vortexed and incubated at 21° C. for 5 minutes. These 5 lysates were then added directly to a series of SV mini columns (Promega Corporation, Madison, Wis. cat #Z3111). The first column (#1) was spun at 2,000×g for 2 minutes. To the flow through from column #1, 65 μl 100% ethanol (AAPER Alcohol and Chemical Co., Shelbyville, Ky.) was added and vortexed thoroughly. This lysate at 25% ethanol was added directly to SV mini column #2 and spun at 2,000×g for 2 minutes. To the flow through from column #2, 260 μl 100% ethanol was added and vortexed thoroughly. This lysate at 50% ethanol was added directly to SV mini column #3 and spun at 2,000×g for 2 minutes. All SV mini column membranes were rinsed twice with 500 μl aliquots of 80% ethanol (v/v with water) and spun at 2,000×g. A final spin at 8,000×g for 5 minutes removed trace ethanol from the column membrane. A 50 μl aliquot of nanopure water was added directly to the membrane of each column and incubated at 21° C. for 5 minutes. The eluate was captured in a fresh tube by spinning the column at 8,000×g for 2 minutes.

A 5 μl sample of the eluate from each column was mixed with 5 μl of 2× formamide loading dye (Ambion™, Austin, Tex.) and heated at 80° C. for 3 minutes. This mixture was then loaded on a 1×TBE pH 8.3 (89 mM Tris, 89 mM boric acid, 20 mM EDTA)/8 M Urea, 15% Polyacrylamide gel (Invitrogen, Carlsbad, Calif.). Marker lanes contain 100 b-500 b markers (Ambion cat #7140). Electrophoresis was performed at 100 volts (constant) for 2 hours at 21° C. The gel was removed from the plastic cassette and placed in a solution of 50 ml 1×TBE pH 8.3 buffer plus a 5 μl aliquot of Sybr Gold (Invitrogen) and stained for 5 minutes with occasional mixing. The gel was digitally imaged using the Amersham (Piscataway, N.J.) Typhoon platform with settings of: (1) ex 488/em 526. (2) PMT 450.

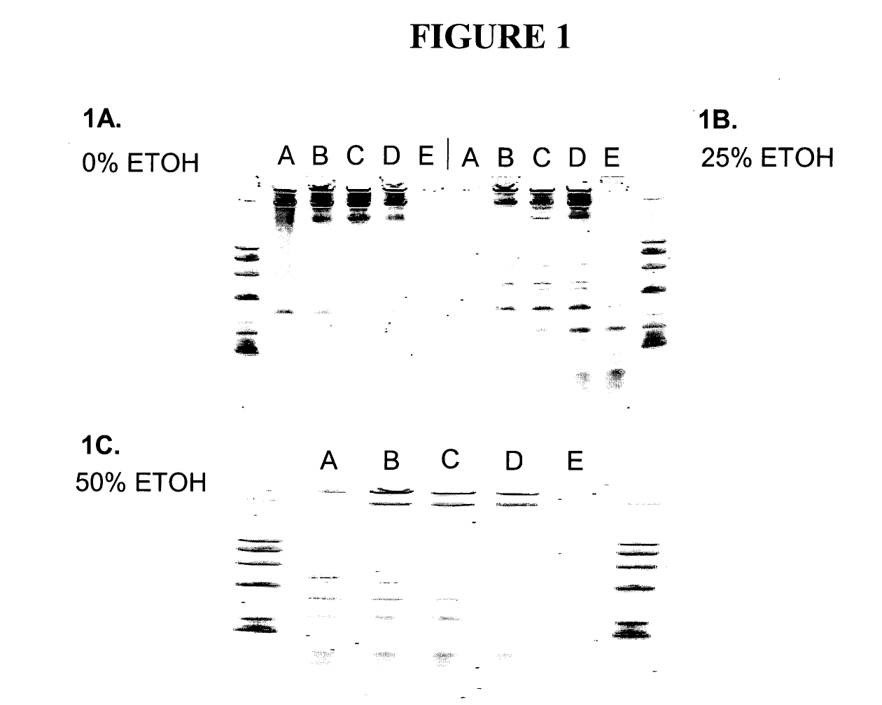

Digital images of the gels are shown in FIGS. 1A-1C. For each gel, the GITC to Urea ratio for each lane is as follows: Lane A: 175/0; Lane B: 130/45; Lane C: 85/90; Lane D: 45/130; and Lane E: 0/175. FIG. 1A shows the RNA from samples that were in 0% ethanol when passed through the SV mini column; FIG. 1B shows the RNA from the samples that were in 25% ethanol when passed through the SV mini column; and FIG. 1C shows the RNA from the samples that were in 50% ethanol when passed through the SV mini column. These figures shows that, unexpectedly, the use of urea as the chaotropic agent in about 25% ethanol, without using GITC, allows small RNAs to be purified, without also purifying large RNAs, as shown in lane E in FIG. 1B.

### Example 2

### Single Membrane Small RNA Purification with Various Concentrations of NaCl

This example describes the small RNA purification from a cell lysate using urea, a compaction agent, and various concentrations of NaCl. The purifications were accomplished using just a single binding column membrane without using a separate lysate purification step. Nine tubes, each containing 1×10⁶ cultured 293T human cells were centrifuged at 8,000×g and rinsed twice with 500 μl 1×PBS pH 6.8 to remove cell culture media. PBS supernatant was removed after each centrifugation of cells. To each tube was added: (1) 360 μl 8 M Urea, 20 mM TRIS pH 7.5, and (2) 60 μl 250 mM Hexamminecobalt(III)chloride (Sigma #H-7891) in 1×TE pH8.0. 5 M NaCl and 20 mM TRIS pH 7.5 was added to tube 0-8 in variable amounts as follows: Tube 0: 0 μl NaCl+125 μl, Tube 1:3.5 μl NaCl+121.5 μl TRIS, Tube 2: 5 μl NaCl+120 μl, Tube 3: 10 μl NaCl+115 μl TRIS, Tube 4: 25 μl NaCl+100 μl TRIS, Tube 5: 50 μl NaCl+75 μl TRIS, Tube 6: 75 μl NaCl+50 μl TRIS, Tube 7: 100 μl NaCl+25 μl TRIS, Tube 8: 125 μl NaCl+0 μl TRIS.

All tubes were seeded with a 10 aliquot of a 1:100 dilution of a T7 RNA synthesis reaction: small T7 plasmid runoff ssRNA fragments (25 b, 45 b, and 70 b) produced using the T7 Ribomax Express system (Promega cat #P1700) and restriction enzyme digested plasmids (pGEM-3zf(+) cat #P2271, and pGEM-5zf(+) cat #P2241, Promega). Tubes were vortexed thoroughly and incubated at 21° C. for 5 minutes. 180 μl of 100% ethanol was added to each tube for a final volume of 725 μl. The final concentration of NaCl in the binding solution was variable: i.e. tube 0: 0 mM tube 1: 24 mM, tube 2: 34 mM, tube3: 69 mM, tube 4: 172 mM, tube 5: 345 mM, tube 6: 517 mM, tube 7: 690 mM, and tube 8: 862 mM.

These lysates at 25% ethanol and variable concentrations of NaCl were added directly to individual SV mini columns (Promega) and spun at 2,000×g for 2 minutes. The flow through was discarded. All SV mini columns membranes were rinsed twice with 500 μl aliquots of 80% ethanol (v/v with water) and spun at 2,000×g. A final spin at 8,000×g for 5 minutes removed trace ethanol from the column membranes. A 50 μl aliquot of nanopure water was added directly to the membrane of each column and incubated at 21° C. for 5 minutes. The eluate was captured in a fresh tube by spinning the column at 8,000×g for 2 minutes. A 5 μl sample of the eluate from each column was mixed with 5 μl of 2× formamide loading dye (Ambion) and heated at 80° C. for 3 minutes. This mixture was then loaded on a 1×TBE pH 8.3/8 M urea, 15% polyacrylamide gel (Invitrogen). One Marker lane contained 100 b-500 b markers (Ambion). An additional marker lane (25 b, 45 b, and 70 b) contained small T7 runoff transcripts produced from restriction enzyme digested plasmids (pGEM-3zf(+) cat #P2271, and pGEM-5zf(+) cat #P2241). Electrophoresis was performed at 100 volts (constant) for 2 hours at 21° C. The gel was removed from the plastic cassette and placed in a solution of 50 ml 1×TBE pH 8.3 buffer plus a 5 μl aliquot of Sybr Gold (Invitrogen) and stained for 5 minutes with occasional mixing. The gel was digitally imaged using the Amersham Typhoon platform with settings of: (1) ex 488/em 526. (2) PMT 450.

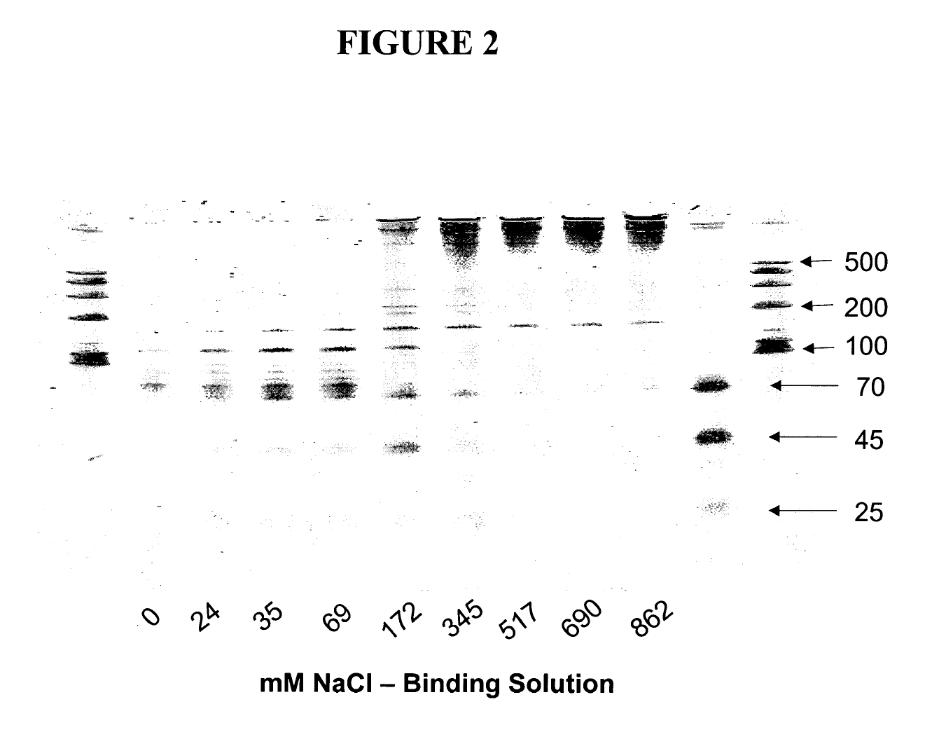

A digital image of the gel is shown in FIG. 2. The NaCl concentration for each lane is shown at the bottom of the gel. As can be seen in this figure: 1) an absence of added NaCl showed a reduced level of binding of small RNA (e.g. less than about 100 bases) to the membrane; 2) increasing levels of NaCl, from about 24 to 345 mM, in the binding solutions produced binding of small RNAs (e.g. 25 bases, 45 bases, and less than about 100 bases); and 3) increasing levels of NaCl above 345 mM in binding solutions yielded larger RNA molecules and less of the small RNA molecules.

### Example 3

### Single Membrane Small RNA Purification with Various Concentrations of Hexamminecobalt(III)chloride

This example describes the small RNA purification from a cell lysate using urea and various concentrations of compaction agent Hexamminecobalt(III)chloride. The purifications were accomplished using just a single binding column membrane without using a separate lysate purification step. Nine tubes, each containing 1×10⁶ cultured 293T human cells were rinsed twice with 500 μl 1×PBS pH 6.8 to remove cell culture media. PBS supernatant was removed after centrifugation of cells. To each tube was added: (1) 175 μl 8 M Urea, 20 mM TRIS pH 7.5 and (2) 5 μl 5 M NaCl, 300 mM Hexamminecobalt(III)chloride (HACC) (in 1×TE pH 8.0) and 20 mM TRIS pH 7.5 was added in variable amounts to tube 0-8 as follows: Tube 0: 0 μl HACC+95 μl TRIS, Tube 1: 2.5 μl HACC+93.5 μl TRIS, Tube 2: 5 μl HACC+90 μl TRIS, Tube 3: 7.5 μl HACC+87.5 μl TRIS, Tube 4: 10 μl HACC+85 μl TRIS, Tube 5: 12.5 μl HACC+82.5 μl TRIS, Tube 6: 15 μl HACC+80 μA TRIS, Tube 7: 17.5 μl HACC+77.5 μl TRIS. All tubes were seeded with 3 μl of a 1:100 dilution of a T7 RNA synthesis reaction. Small T7 plasmid runoff ssRNA fragments (25 b, 45 b, and 70 b) produced using the T7 Ribomax Express system (Promega) and restriction enzyme digested plasmids (pGEM-3zf(+) cat #P2271, and pGEM-5zf(+) cat #P2241). Tubes were vortexed thoroughly and incubated at 21° C. for 5 minutes. 100 μl of 100% ethanol was added to each tube for a final volume of 375 μl. The final concentration of Hexamminecobalt(III)chloride in the binding solution was varied at: 0 mM, 2 mM 4 mM, 6 mM, 8 mM, 10 mM, 12 mM, and 14 mM. These lysates at 27% ethanol and variable concentrations of Hexamminecobalt(III) chloride was added directly to individual SV mini columns (Promega) and spun at 2,000×g for 2 minutes. The flow through was discarded. All SV mini columns membranes were rinsed twice with 500 μl aliquots of 80% ethanol (v/v with water) and spun at 2,000×g. A final spin at 8,000×g for 5 minutes removed trace ethanol from the column membrane. A 50 μl aliquot of nanopure water was added directly to the membrane of each column and incubated at 21° C. for 5 minutes. The eluate was captured in a fresh tube by spinning the column at 8,000×g for 2 minutes. A 5 μl sample of the eluate from each column was mixed with 5 μl of 2× formamide loading dye (Ambion) and heated at 80° C. for 3 minutes. This mixture was then loaded on a 1×TBE pH 8.3/8 M urea, 15% polyacrylamide gel (Invitrogen). One Marker lane contained 100 b-500 b markers (Ambion). An additional marker lane (25 b, 45 b, and 70 b) contained small T7 runoff transcripts produced from restriction enzyme digested plasmids (pGEM-3zf(+) cat #P2271, and pGEM-5zf(+) cat # P2241). Electrophoresis was performed at 100 volts (constant) for 2 hours at 21° C. The gel was removed from the plastic cassette and placed in a solution of 50 ml 1×TBE pH 8.3 buffer plus a 5 μl aliquot of Sybr Gold (Invitrogen) and stained for 5 minutes with occasional mixing. The gel was digitally imaged using the Amersham Typhoon platform with settings of: (1) ex 488/em 526. (2) PMT 450.

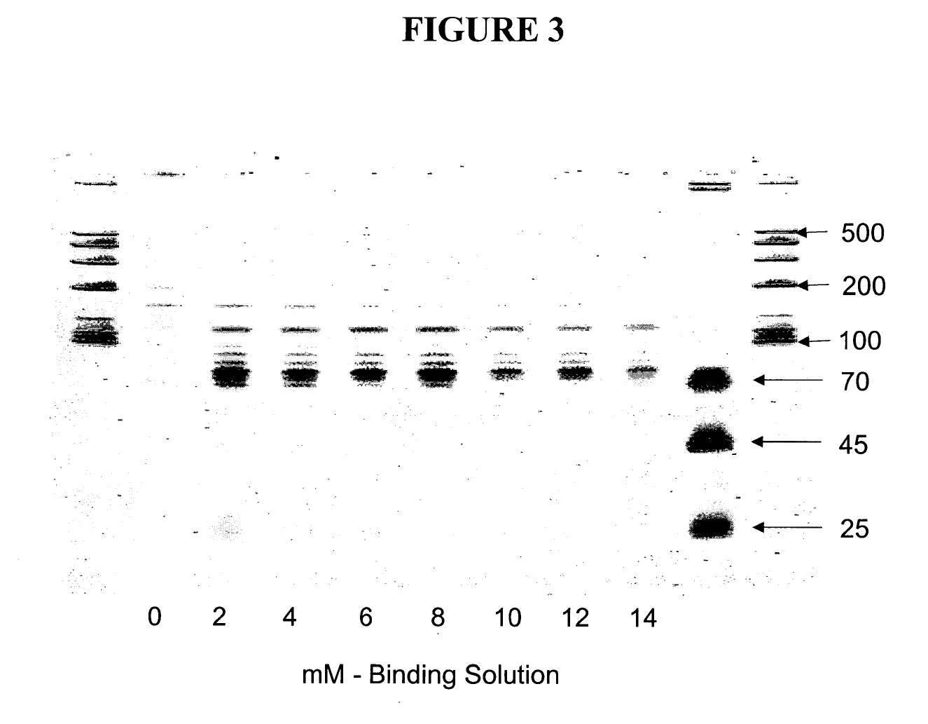

A digital image of the gel is shown in FIG. 3. The HACC concentration for each lane is shown at the bottom of the gel. As can be seen in this figure: 1) an absence of added HACC showed very little small RNA binding; 2) concentrations of HACC between 2 mM and 8 mM showed high levels of small RNA binding; and 3) concentrations of HACC above 8 mM showed reduced levels of binding of small RNA molecules.

### Example 4

### Single Membrane Small RNA Purification from Yeast Cells

This example describes the small RNA purification from a yeast cell lysate. The purifications were accomplished using just a single binding column membrane without using a separate lysate purification step. Yeast cells (ATCC 200528) were cultured overnight in 5 ml of YPD media in a 15 ml plastic culture tube at 30° C., shaken at 250 rpm. Absorbance was measured at 600 nm. Yeast cells were rinsed twice with 1×TE pH 8.0 to remove cell culture media. Yeast cells at 600 nm optical densities of 0.6, 1.2, 1.7, and 2.3 were added to separate tubes and spun at 8,000×g for 5 minutes. TE supernatant was removed from tubes. Cells were incubated with 50 units lyticase (Sigma) in 20 μl 1×TE pH 8.0 plus 3 μl 48.7% BME (betamercaptoethanol) for 2 hours at 30° C. To each tube was added 200 μl of a mixture containing 8 M urea, 20 mM TRIS pH 7.5, 125 mM NaCl, and 25 mM Hexamminecobalt(III)chloride. Tubes were vortexed thoroughly and incubated at 21° C. for 5 minutes. 530 μl 75% ethanol (v/v water) was added to each tube for a final volume of 750 μl.

This lysate was added directly to individual SV mini columns (Promega) and spun at 2,000×g for 2 minutes. The flow through was discarded. All SV mini columns membranes were rinsed twice with 500 μl aliquots of 80% ethanol (v/v with water) and spun at 2,000×g. A final spin at 8,000×g for 5 minutes removed trace ethanol from the column membrane. A 50 μl aliquot of nanopure water was added directly to the membrane of each column and incubated at 21° C. for 5 minutes. The eluate was captured in a fresh tube by spinning the column at 8,000×g for 2 minutes.

A 5 μl sample of the eluate from each column was mixed with 5 μl of 2× formamide loading dye (Ambion) and heated at 80° C. for 3 minutes. This mixture was then loaded on a TBE pH 8.3/8 M urea, 15% polyacrylamide gel (Invitrogen). One Marker lane contained 100 b-500 b markers (Ambion). An additional marker lane (25 b, 45 b, and 70 b) contained small T7 runoff transcripts produced from restriction enzyme digested plasmids (pGEM-3zf(+) cat #P2271, and pGEM-5zf(+) cat #P2241). Electrophoresis was performed at 100 volts (constant) for 2 hours at 21° C. The gel was removed from the plastic cassette and placed in a solution of 50 ml 1×TBE pH 8.3 buffer plus a 5 μl aliquot of Sybr Gold (Invitrogen) and stained for 5 minutes with occasional mixing. The gel was digitally imaged using the Amersham Typhoon platform with settings of: (1) ex 488/em 526. (2) PMT 450.

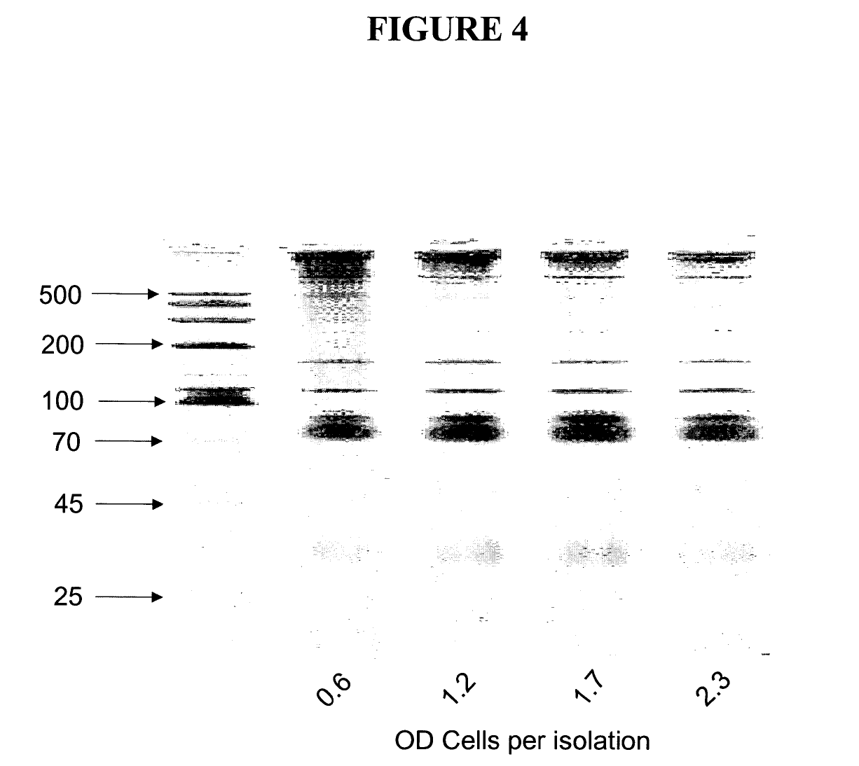

A digital image of the gel is shown in FIG. 4. The optical densities of yeast cells for each lane is shown at the bottom of the gel. As can be seen in this figure, small RNA molecules were able to be purified from yeast cells using the methods described in this example.

### Example 5

### Single Membrane Small RNA Purification from E. coli Cells

This example describes the small RNA purification from a cell lysate of E. coli cells using just a single binding column membrane without using a separate lysate purification step. 50 ml cultures of E. coli strains JM109 or JM109 (pUC18) were cultured overnight in LB media at 37° C. with shaking at 250 rpm. Bacterial culture volumes of 25 μl, 50 μl, 100 μl, 250 μl, and 500 μl were added to separate tubes and spun at 8,000×g for 5 minutes. Bacterial cells were rinsed twice with 1×TE pH 8.0 to remove cell culture media. TE supernatant was removed. To each tube was added 200 μl of a mixture containing 8 M urea, 20 mM TRIS pH 7.5, 125 mM NaCl, and 25 mM Hexamminecobalt(III)chloride.

Tubes were vortexed thoroughly and incubated at 21° C. for 5 minutes. 530 μl 75% ethanol (v/v water) was added to each tube for a final volume of 730 μl. This lysate was added directly to individual SV mini columns (Promega) and spun at 2,000×g for 2 minutes. The flow through was discarded. All SV mini columns membranes were rinsed twice with 500 μl aliquots of 80% ethanol (v/v with water) and spun at 2,000×g. A final spin at 8,000×g for 5 minutes removed trace ethanol from the column membrane. A 50 μl aliquot of nanopure water was added directly to the membrane of each column and incubated at 21° C. for 5 minutes. The eluate was captured in a fresh tube by spinning the column at 8,000×g for 2 minutes. A 5 μl sample of the eluate from each column was mixed with 5 μl of 2× formamide loading dye (Ambion) and heated at 80° C. for 3 minutes. This mixture was then loaded on a 1×TBE pH8.3/8M urea, 15% polyacrylamide gel (Invitrogen). One Marker lane contained 100 b-500 b markers (Ambion). An additional marker lane (25 b, 45 b, and 70 b) contained small T7 runoff transcripts produced from restriction enzyme digested plasmids (pGEM-3zf(+) cat #P2271, and pGEM-5zf(+) cat #P2241). Electrophoresis was performed at 100 volts (constant) for 2 hours at 21° C. The gel was removed from the plastic cassette and placed in a solution of 50 ml 1×TBE pH 8.3 buffer plus a 5 μl aliquot of Sybr Gold (Invitrogen) and stained for 5 minutes with occasional mixing. The gel was digitally imaged using the Amersham Typhoon platform with settings of: 1. ex 488/em 526. 2. PMT 450.

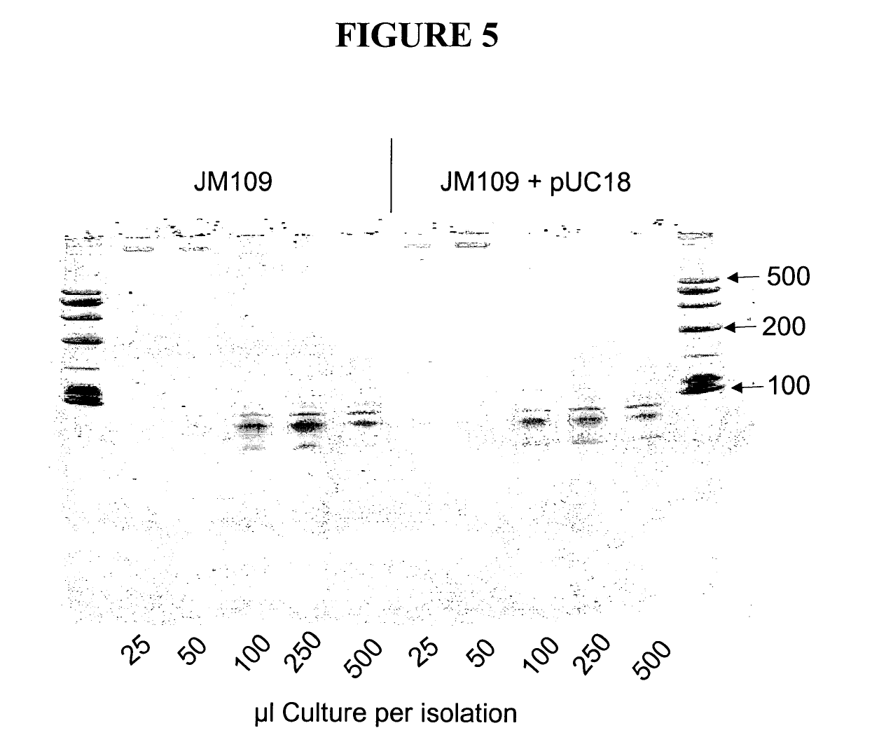

A digital image of the gel is shown in FIG. 5. As can be seen in this figure: 1) culture volumes of 25-50 ul show low yields of RNA for E. coli cells, and 2) a culture volume of 100 ul or more shows a good yield of small RNA molecules (e.g. RNA molecules less than about 100 bases).

### Example 6

### Single Membrane Small RNA Purification with Various Buffers at Various pHs

This example describes the small RNA purification from a human cell lysate using urea, a compaction agent, and various buffers at various pHs. The purifications were accomplished using just a single binding column membrane without using a separate lysate purification step. 1×10⁶ cultured 293T human cells were rinsed twice with 500 μl 1×PBS pH 6.8 to remove cell culture media. PBS supernatant was removed after centrifugation of cells. Separate solutions of 8 M Urea were prepared at 20 mM MES (2-morpholinoethane sulfonic acid) pH5.5, 20 mM MES pH 6.0, 20 mM TRIS pH 8.3, 20 mM TRIS pH 9.0, 20 mM Sodium Citrate pH 5.5, 20 mM Sodium Citrate pH 6.0, 20 mM HEPES (4-(2-hydroxyethyl)piperazine-1-ethanesulfonic acid) pH 7.5, and 20 mM HEPES pH 8.0. To each tube was added: (1) 175 μl 8 M Urea with differing pH and buffer, as described above; and (2) 5 μl 5 M NaCl (3) 20 μl 300 mM Hexamminecobalt(III)chloride in 1×TE pH8.0. All tubes were seeded with 3 μl of a 1:100 dilution of a T7 RNA synthesis reaction. Small T7 plasmid runoff ssRNA fragments (25 b, 45 b, and 70 b) produced using the T7 Ribomax Express system (Promega) and restriction enzyme digested plasmids (pGEM-3zf(+) cat #P2271, and pGEM-5zf(+) cat #P2241). Tubes were vortexed thoroughly and incubated at 21° C. for 5 minutes. 200 μl of 100% ethanol was added to each tube for a final volume of 400 μl. These lysates at differing pH levels were added directly to individual SV mini columns (Promega) and spun at 2,000×g for 2 minutes. The flow through was discarded. All SV mini column membranes were rinsed twice with 500 μl aliquots of 80% ethanol (v/v with water) and spun at 2,000×g. A final spin at 8,000×g for 5 minutes removed trace ethanol from the column membrane. A 50 μl aliquot of nanopure water was added directly to the membrane of each column and incubated at 21° C. for 5 minutes. The eluate was captured in a fresh tube by spinning the column at 8,000×g for 2 minutes. A 5 μl sample of the eluate from each column was mixed with 5 μl of 2× formamide loading dye (Ambion) and heated at 80° C. for 3 minutes. This mixture was then loaded on a 1×TBE pH 8.3/8M urea, 15% polyacrylamide gel (Invitrogen). One Marker lane contained 100 b-500 b markers (Ambion). An additional marker lane (25 b, 45 b and 70 b) contained small T7 runoff transcripts produced from restriction enzyme digested plasmids (pGEM-3zf(+) cat #P2271, and pGEM-5zf(+) cat #P2241). Electrophoresis was performed at 100 volts (constant) for 2 hours at 21° C. The gel was removed from the plastic cassette and placed in a solution of 50 ml 1×TBE pH 8.3 buffer plus a 5 μl aliquot of Sybr Gold (Invitrogen) and stained for 5 minutes with occasional mixing. The gel was digitally imaged using the Amersham Typhoon platform with settings of: (1) ex 488/em 526. (2) PMT 450.

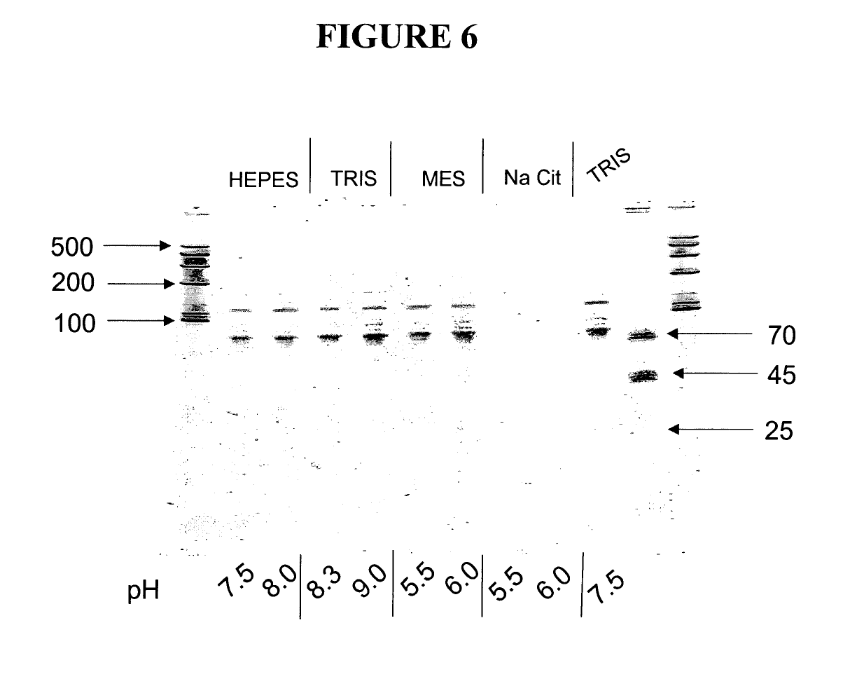

A digital image of the gel is shown in FIG. 6. As can be seen in this figure: 1) the HEPES, MES, and TRIS buffers allowed the SV membrane to capture small RNA fragments (e.g. less than about 200 bases); and 2) pH ranges from 5.5 to 9.0 produced similar yields of small RNA molecules.

### Example 7

### Single Membrane Small RNA Purification from Cultured Human Cells with Various Chaotropes

This example describes the small RNA purification from a human cell lysate using a compaction agent and various chaotropic agents. The purifications were accomplished using just a single binding column membrane without using a separate lysate purification step. 1×10⁶ cultured 293T human cells were washed twice with 200 μl 1×PBS pH6.8 to remove cell culture media. PBS supernatant was removed after centrifugation of cells. Separate solutions of 2M thiourea 20 mM TRIS pH7.5, 4M urethane 115 mM TRIS pH7.5, 9M acetamide 115 mM TRIS pH7.5 were prepared. To each tube was added: 1. 175 μl chaotrope with TRIS buffer. 2. 5 μl 5M NaCl 3. 20 μl 250 mM hexamminecobalt(III) chloride in TE buffer.

All tubes were seeded with 1 μl of a 1:100 dilution of a T7 RNA synthesis reaction. Small T7 plasmid runoff ssRNA fragments (25, 45, and 70b) produced using the T7 Ribomax Express system (Promega) and cut plasmids (pGEM-3zf+, and pGEM-5zf+). Tubes were vortexed well and held at room temperature for 5 minutes. 550 μl of 75% ethanol was added to each tube for a final volume of 750 μl. These lysates with differing chaotropes were added directly to individual SV mini columns (Promega) and centrifuged at 2,000×g for 2 minutes. The flow through was discarded. All SV mini column membranes were washed twice with separate 500 μl aliquots of 75% ethanol and centrifuged at 2,000×g. A final spin at 8,000×g for 5 minutes removed trace ethanol from the column membrane. A 35 μl aliquot of nanopure water was added directly to the membrane of each column and held at room temperature for 5 minutes. The eluate was captured in a fresh tube by spinning the column at 8,000×g for 2 minutes. A 5 μl portion of the eluate from each column was mixed with 5 μl of 2× formamide loading dye (Ambion) and heated at 80° C. for 3 minutes.

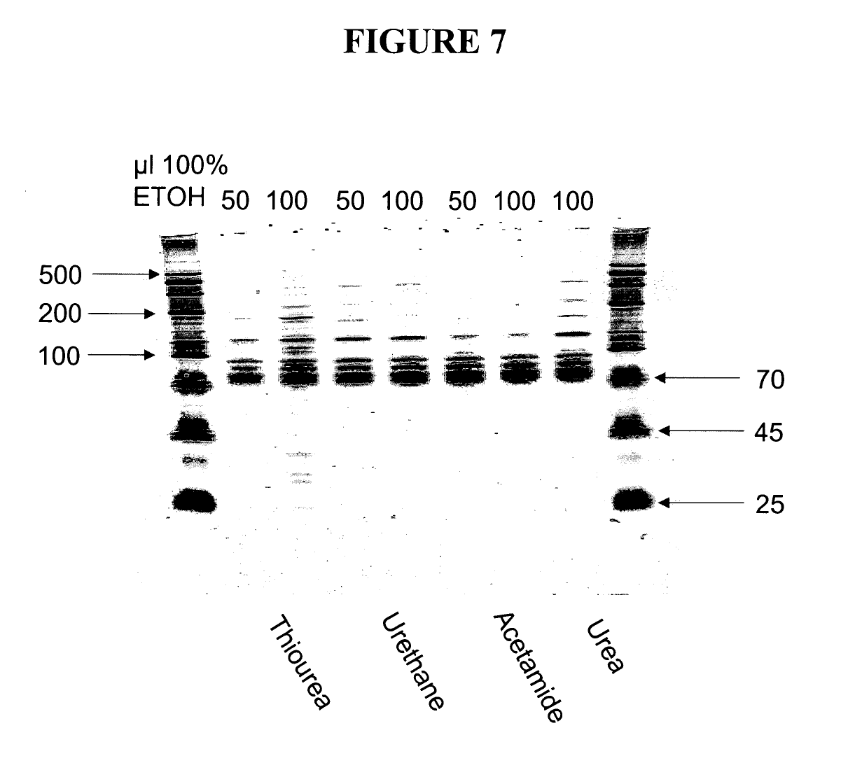

This mixture was then loaded on a 1×TBE/8M urea, 15% polyacrylamide gel (Invitrogen). One Marker lane contained 100-500b markers (Ambion). An additional marker lane (25, 45 and 70b) contained small T7 runoff transcripts produced from cut plasmids (pGEM-3zf+, and pGEM-5zf+). Electrophoresis was performed at 100 volts (constant) for 2 hours at room temperature. The gel was removed from the plastic cassette and placed in a solution of 50 ml 1×TBE buffer plus a 5 μl aliquot of Sybr Gold (Invitrogen) and stained for 5 minutes with occasional mixing. The gel was digitally imaged using the Amersham Typhoon platform with settings of: 1. ex488/em526. 2. PMT 450. As shown in FIG. 7, chaotropes such as thiourea, acetamide, urethane, and urea were suitable for the isolation of small RNA from cultured cells with this system.

### Example 8

### Single Membrane Small RNA Purification from Tissue with Various Chaotropes

This example describes the small RNA purification from beef tissue using a compaction agent and various chaotropic agents. The purifications were accomplished using just a single binding column membrane without using a separate lysate purification step. Beef liver previously frozen at −70° C. was weighed into separate 50 ml conical tubes. Solutions of 2M thiourea 20 mM TRIS pH7.5, 4M urethane 115 mM TRIS pH7.5, 9M acetamide 115 mM TRIS pH7.5, or 8M urea 20 mM TRIS pH7.5 were added to the each of 5 separate tubes so 30 mg of tissue per tube was covered by 1750 of chaotropic solution per tube. Tissue was homogenized mechanically for 2 minutes, in 4×30 second bursts followed by 15 seconds on ice to allowing cooling. Tissue homogenate was centrifuged at 14000×g for 15 minutes. The supernatant was removed to a new tube. To each tube was added: 1. 10 ul or 25 ul tissue homogenate supernatant 2. 165 μl or 150 μl corresponding chaotrope with TRIS buffer. 3. 5 μl 5M NaCl 4. 20 μl 250 mM Hexamminecobalt(III) chloride in TE buffer.

Tubes were vortexed and held at room temperature for 5 minutes. 550 μl of 75% ethanol was added to each tube for a final volume of 750 μl. Each lysate with differing chaotropes was added directly to individual SV mini columns (Promega) and centrifuged at 2,000×g for 2 minutes. The flow through was discarded. All SV mini columns membranes were washed twice with separate 500 μl aliquots of 75% ethanol (v/v with water) and centrifuged at 2,000×g. A final spin at 8,000×g for 5 minutes removed trace ethanol from the column membrane. A 35 μl aliquot of nanopure water was added directly to the membrane of each column and held at room temperature for 5 minutes. The eluate was captured in a fresh tube by spinning the column at 8,000×g for 2 minutes.

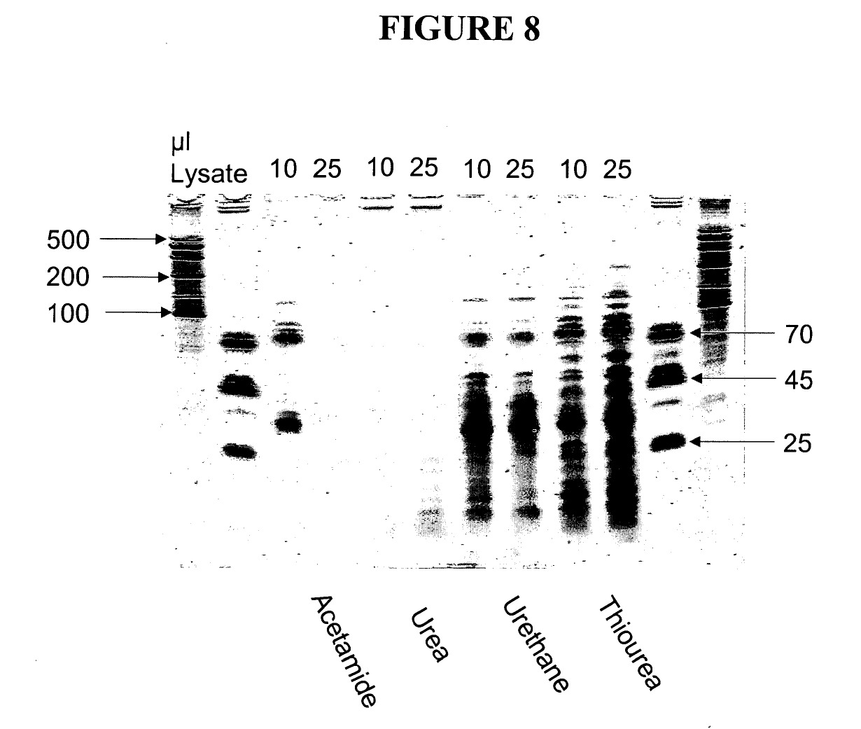

A 5 μl portion of the eluate from each column was mixed with 5 μl of 2× formamide loading dye (Ambion) and heated at 80° C. for 3 minutes. This mixture was then loaded on a 1×TBE/8M urea, 15% polyacrylamide gel (Invitrogen). One Marker lane contained 100-500b markers (Ambion). An additional marker lane (25, 45 and 70b) contained small T7 runoff transcripts produced from cut plasmids (pGEM-3zf+, and pGEM-5zf+). Electrophoresis was performed at 100 volts (constant) for 2 hours at room temperature. The gel was removed from the plastic cassette and placed in a solution of 50 ml 1×TBE buffer plus a 5 μl aliquot of Sybr Gold (Invitrogen) and stained for 5 minutes with occasional mixing. The gel was digitally imaged using the Amersham Typhoon platform with settings of: 1. ex488/em526. 2. PMT 450. As shown in FIG. 8, chaotropes such as thiourea, acetamide, urethane, and urea were suitable for the isolation of small RNA from beef liver tissue with this method.

### Example 9

### Single Membrane Small RNA Purification from Human Cells with Urea and Isopropanol

This example describes the small RNA purification from human cells using urea, a compaction agent, and isopropanol. The purifications were accomplished using just a single binding column membrane without using a separate lysate purification step. 1×10⁶ cultured 293T human cells were washed twice with 200 μl 1×PBS pH 6.8 to remove cell culture media. PBS supernatant was removed after centrifugation of cells. To each of 9 tubes was added 200 μl of a mixture containing 8M urea, 20 mM TRIS pH7.5, 125 mM NaCl, and 25 mM Hexamminecobalt(III)chloride. Tubes were vortexed well and held at room temperature for 5 minutes. All tubes were seeded with 1 μl of a 1:100 dilution of a T7 RNA synthesis reaction. Small T7 plasmid runoff ssRNA fragments (25, 45, and 70b) produced using the T7 Ribomax Express system (Promega) and cut plasmids (pGEM-3zf+, and pGEM-5zf+). Tubes were vortexed well and held at room temperature for 5 minutes. To each separate tube was added either 100, 200, 300, 400, 500, 600, 700, 800, or 900 μl of 100% isopropanol. These lysates at differing isopropanol concentrations were added directly to individual SV mini columns (Promega) and centrifuged at 2,000×g for 2 minutes. The flow through was discarded. All SV mini columns membranes were washed twice with separate 500 μl aliquots of 75% ethanol (v/v with water) and centrifuged at 2,000×g. A final spin at 8,000×g for 5 minutes removed trace ethanol from the column membrane. A 50 μl aliquot of nanopure water was added directly to the membrane of each column and held at room temperature for 5 minutes. The eluate was captured in a fresh tube by spinning the column at 8,000×g for 2 minutes.

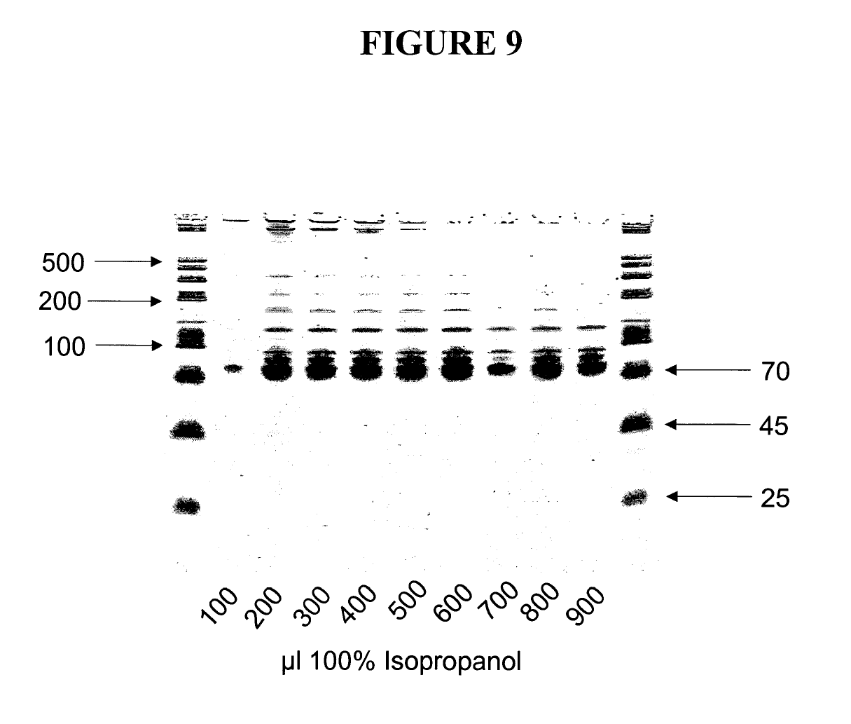

A 5 μl portion of the eluate from each column was mixed with 50 of 2× formamide loading dye (Ambion) and heated at 80° C. for 3 minutes. This mixture was then loaded on a 1×TBE/8M urea, 15% polyacrylamide gel (Invitrogen). One Marker lane contained 100-500b markers (Ambion). An additional marker lane (25, 45 and 70b) contained small T7 runoff transcripts produced from cut plasmids (pGEM-3zf+, and pGEM-5zf+). Electrophoresis was performed at 100 volts (constant) for 2 hours at room temperature. The gel was removed from the plastic cassette and placed in a solution of 50 ml 1×TBE buffer plus a 5 μl aliquot of Sybr Gold (Invitrogen) and stained for 5 minutes with occasional mixing. The gel was digitally imaged using the Amersham Typhoon platform with settings of: 1. ex488/em526. 2. PMT 450. As shown in FIG. 9, small RNA was able to be purified in the presence of isopropanol, with larger volumes of isopropanol yielding more purified (less larger RNA sequence) RNA samples.

### Example 10

### Single Membrane Small RNA Purification from Human Cells with Urea and Methanol

This example describes the small RNA purification from human cells using urea, a compaction agent, and methanol. The purifications were accomplished using just a single binding column membrane without using a separate lysate purification step. 1×10⁶ cultured 293T human cells were washed twice with 200 μl 1×PBS pH 6.8 to remove cell culture media. PBS supernatant was removed after centrifugation of cells. To each of 9 tubes was added 200 μl of a mixture containing 8M urea, 20 mM TRIS pH7.5, 125 mM NaCl, and 25 mM Hexamminecobalt(III)chloride. Tubes were vortexed well and held at room temperature for 5 minutes. All tubes were seeded with 1 μl of a 1:100 dilution of a T7 RNA synthesis reaction. Small T7 plasmid runoff ssRNA fragments (25, 45, and 70b) produced using the T7 Ribomax Express system (Promega) and cut plasmids (pGEM-3zf+, and pGEM-5zf+). Tubes were vortexed well and held at room temperature for 5 minutes. To each of separate tubes was added 100, 200, 300, 400, 500, 600, 700, 800, or 900 μl of 100% methanol. Each lysate at differing methanol concentrations was added directly to individual SV mini columns (Promega) and centrifuged at 2,000×g for 2 minutes. The flow through was discarded. All SV mini columns membranes were washed twice with separate 500 μl aliquots of 75% ethanol (v/v with water) and centrifuged at 2,000×g. A final spin at 8,000×g for 5 minutes removed trace ethanol from the column membrane. A 50 μl aliquot of nanopure water was added directly to the membrane of each column and held at room temperature for 5 minutes. The eluate was captured in a fresh tube by spinning the column at 8,000×g for 2 minutes.

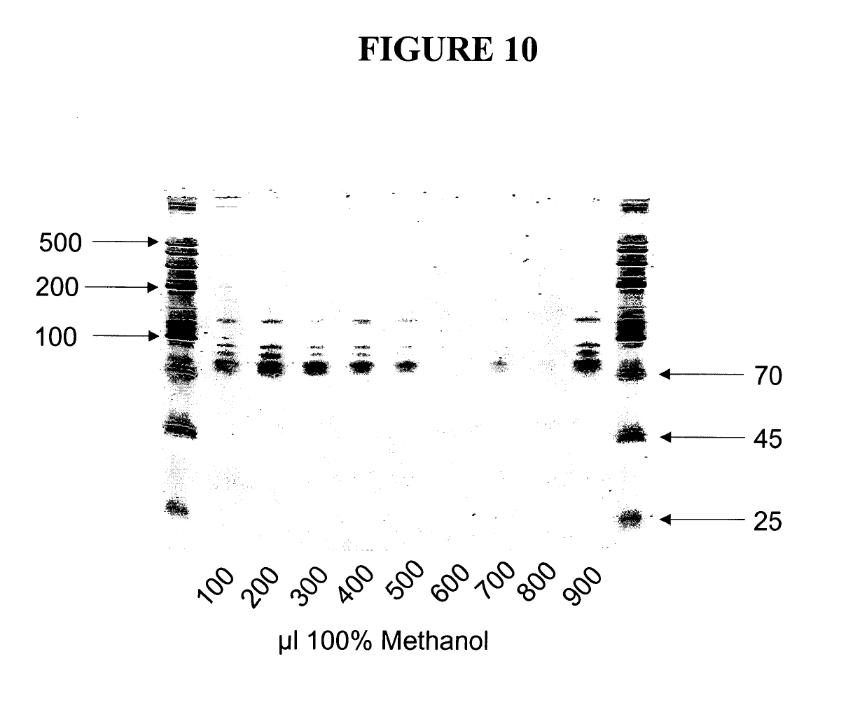

A 5 μl portion of the eluate from each column was mixed with 5 μl of 2× formamide loading dye (Ambion) and heated at 80° C. for 3 minutes. This mixture was then loaded on a 1×TBE/8M urea, 15% polyacrylamide gel (Invitrogen). One Marker lane contained 100-500b markers (Ambion). An additional marker lane (25, 45 and 70b) contained small T7 runoff transcripts produced from cut plasmids (pGEM-3zf+, and pGEM-5zf+). Electrophoresis was performed at 100 volts (constant) for 2 hours at room temperature. The gel was removed from the plastic cassette and placed in a solution of 50 ml 1×TBE buffer plus a 5 μl aliquot of Sybr Gold (Invitrogen) and stained for 5 minutes with occasional mixing. The gel was digitally imaged using the Amersham Typhoon platform with settings of: 1. ex488/em526. 2. PMT 450. As shown in FIG. 10, small RNA was able to be purified in the presence of methanol, with larger volumes of methanol yielding more purified (less larger RNA sequence) RNA samples.

### Example 11

### Single Membrane Small RNA Purification from Plant Tissue with Various Chaotropes

This example describes the small RNA purification from plant tissue using a compaction agent and various chaotropic agents. The purifications were accomplished using just a single binding column membrane without using a separate lysate purification step. Canola was grown for 35 days under fluorescent table top lights with 12 hours of light and 12 hours of darkness per day. Separate solutions of 1: 8M urea 20 mM TRIS pH7.5 125 mM NaCl 25 mM Hexamminecobalt(III)chloride 2: 4M urethane 115 mM TRIS pH7.5 125 mM NaCl 25 mM Hexamminecobalt(III)chloride and 3: 9M acetamide 115 mM TRIS pH7.5 125 mM NaCl 25 mM Hexamminecobalt(III)chloride were added to the each of 3 separate tubes so 30 mg of tissue per tube was covered by 1 ml of chaotropic solution per tube. A 10 μl aliquot of 48.7% β-mercaptoethanol (Promega cat #Z5231) was added to each tube. Tissues were homogenized mechanically for 1.5 minutes, in 3×30 second bursts followed by 15 seconds on ice to allowing cooling. The tissue homogenates were centrifuged at 14,000×g for 5 minutes. The supernatant was removed to a new tube. To each tube was added: 1. 10 μl, 25 μl, or 50 μl tissue homogenate supernatant 2. 190 μl, 175 μl, or 150 μl corresponding chaotrope with TRIS buffer, NaCl, and hexamminecobalt(III) chloride. Tubes were vortexed and held at 21° C. for 5 minutes. Then 5500 of 75% ethanol was added to each tube for a final volume of 750 μl. Each lysate with differing chaotrope was added directly to individual SV mini columns (Promega) and centrifuged at 2,000×g for 2 minutes. The flow through was discarded. All SV mini columns were washed twice with separate 500 μl aliquots of 75% ethanol (v/v with water) and centrifuged at 2,000×g. A final spin at 8,000×g for 5 minutes removed trace ethanol from the column membrane. A 35 μl aliquot of nanopure water was added directly to the membrane of each column and held at 21° C. for 5 minutes. The eluate was captured in a fresh tube by spinning the column at 8,000×g for 2 minutes.

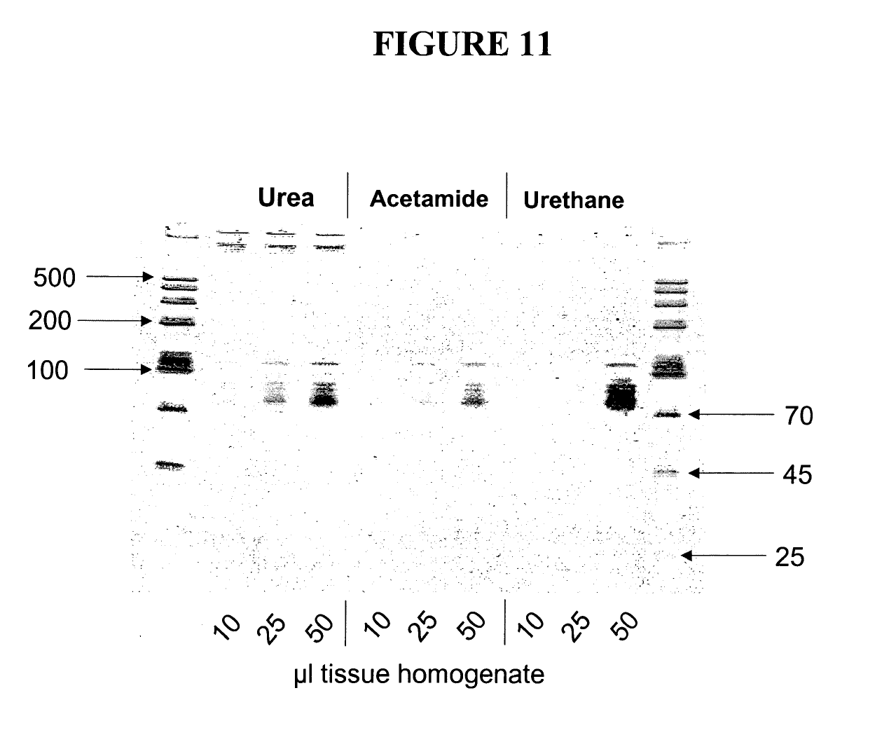

A 5 μl portion of the eluate from each column was mixed with 5 μl of 2× formamide loading dye (Ambion) and heated at 80° C. for 3 minutes. This mixture was then loaded on a 1×TBE/8M urea, 15% polyacrylamide gel (Invitrogen). One Marker lane contained 100-500b markers (Ambion) with runoff transcripts of 25, 60, and 70 bases added. Electrophoresis was performed at 100 volts (constant) for 2 hours at room temperature. The gel was removed from the plastic cassette and placed in a solution of 50 ml 1×TBE buffer plus a 5 μl aliquot of Sybr Gold (Invitrogen) and stained for 5 minutes with occasional mixing. The gel was digitally imaged using the Amersham Typhoon platform with settings of: 1. ex488/em526. 2. PMT 450. As shown in FIG. 11, chaotropes such as urea, acetamide, urethane, and urea were suitable for isolation of small RNA from plant tissue.

### Example 12

### Purification of small RNA with Acetamide and Either No Alcohol or Various Concentrations of Alcohol

In this example, the purification of small RNA from a mixture of RNA using acetamide and either no alcohol or various concentrations of alcohol has been described. In a plastic tube, 5 μl of bovine tRNA 1 μg per ml (Promega part #Y209) and 100 μl 50 bp DNA Step ladder (Promega cat #G4521) were combined. To this mixture was added: (1) 50 μl 5M NaCl, (2) 200 μl 250 mM hexamminecobalt(III)chloride in TE buffer and (3) 1.75 ml of 9M acetamide 115 mM TRIS pH7.5. The tube was vortexed well and held at 21° C. for 5 minutes. Then 200 μl samples were placed into each of 10 sterile 1.5 ml plastic tubes. To one tube no ethanol was added. To the other nine tubes, the following amounts of 95% ethanol/water were added: 30 μl, 50 μl, 65 μl, 80 μl, 90 μl, 100 μl, 125 μl, 150 μl, and 200 μl of 95% ethanol/water per tube, and the tubes were vortexed. About 45% of the additive volume of each sample was added to a SV column (Promega) nested in a 1.5 ml tube, and about 45% of the additive volume of each sample was added to a nylon column (Corning Costar Spin-X catalog #8169) nested in a 1.5 ml tube. Note that the actual final volume of an alcohol-water mixture was less than the additive volume amounts under these conditions. These samples were added to appropriately labeled columns/tubes for each of the above mentioned mixtures. All column/tubes were centrifuged at 8,000×g for 2 minutes. All SV columns were removed to fresh tubes and each was eluted with 25 μl of nuclease free water, added directly to the membrane. Similarly, nylon column/tubes were eluted with 15 ul nuclease free water. The eluate was captured by spinning the tubes at 8,000×g for 2 minutes.

A 10 μl portion of the eluate from each sample was loaded on a 1×TBE/8M urea, 15% polyacrylamide gel (Invitrogen). One Marker lane contained 0.5 μl 50 bp DNA step ladder, and another contained 0.5 μl of bovine tRNA. Electrophoresis was performed at 120 volts (constant) for 2 hours at room temperature. The gel was removed from the plastic cassette and placed in a solution of 50 ml 1×TBE buffer plus a 5 μl aliquot of Sybr Gold (Invitrogen) and stained for 15 minutes with occasional mixing. The gel was digitally imaged using the Amersham Typhoon platform with settings of: 1. ex488/em526. 2. PMT 450.

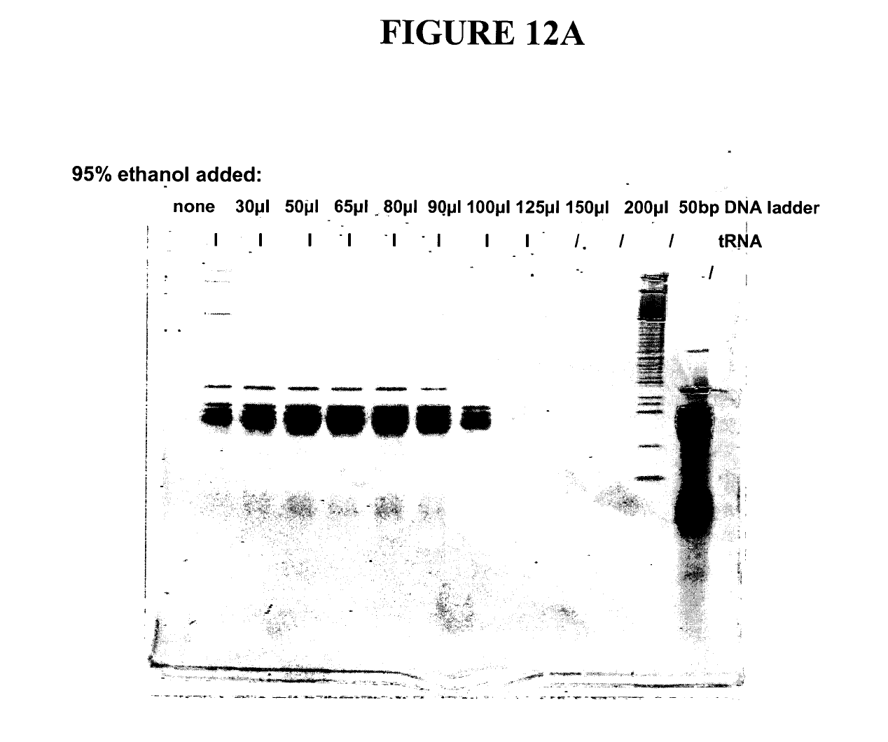

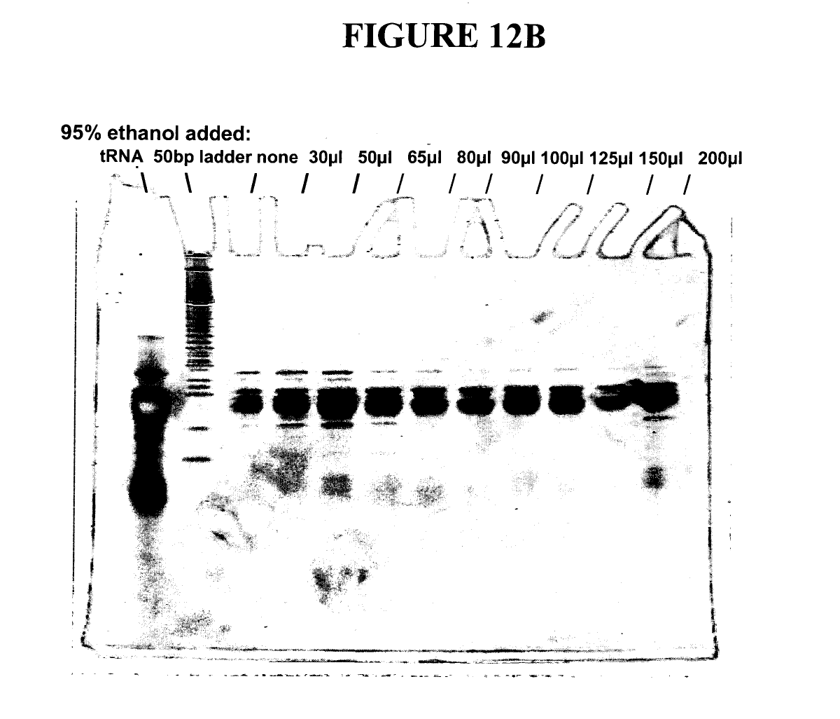

As shown in FIG. 12A (SV membranes) and FIG. 12B (nylon membranes), this method was suitable for evaluating matrix materials for their utility as a binding matrix for this purification method. Further, the method also allowed for the approximate determination of the preferred amount of alcohol to be added, to provide preferred purification of small RNA molecules. Note that the use of alcohol was not required for either matrix tested (see lane 1 of FIG. 12A and lane 3 of FIG. 12B) to purify small RNA molecules from a mixture of RNA and DNA molecules. The test method also showed that RNA was preferentially purified from a mixture of RNA and DNA, even from small DNA molecules such as the 50 base pair DNA molecules.

### Example 13

### Purification of Small RNA with Acetamide and Either No Alcohol or Various Concentrations of Alcohol Using Various Membranes

In this example, the purification of small RNA from a mixture of RNA using acetamide and either no alcohol or various concentrations of alcohol and various membranes has been described. In a plastic tube, 5 μl of bovine tRNA 1 μg per ml (Promega part #Y209), 30 μl of nuclease free water, and 30 μl RNA Markers (Promega cat #G3191) were combined. A 2 μl sample was removed for use as a gel marker (for use on three gel lanes, see below). To this 63 μl mixture was added: (1) 75 μl 5M NaCl, (2) 300 μl 250 mM hexamminecobalt(III) chloride in TE buffer and (3) 2.63 ml of 9M acetamide 115 mM TRIS pH7.5. The tube was vortexed well and held at 21° C. for 5 minutes. Then 300 μl samples were placed into each of 9 sterile 1.5 ml plastic tubes. To one tube no ethanol was added. To the other eight tubes, the following amounts of 95% ethanol were added: 30 μl, 50 μl, 65 μl, 80 μl, 90 μl, 100 μl, 125 μl, and 150 μl of 95% ethanol per tube, and the tubes were vortexed. About 30% of the additive volume of each sample was added to a SV column (Promega) nested in a 1.5 ml tube, and about 45% of the additive volume of each sample was added to a nylon column (Corning Costar Spin-X catalog #8169) nested in a 1.5 ml tube. Note the volumes used were (0 μl) 91 μl, (30 μl) 100 μl, (50 μl) 105 μl, (65 μl) 110 μl, (80 μl) 115 μl, (90 μl) 120 μl, (100 μl) 120 μl, (125 μl) 128 μl, and (150 μl) 135 μl per column/tube. These samples were added to appropriately labeled columns/tubes (SV, nylon or cellulose acetate) for each of the above mentioned mixtures. All column/tubes were centrifuged at 8,000×g for 2 minutes. All SV columns were removed to fresh tubes and each was eluted with 25 μl of nuclease free water, added directly to the membrane. Similarly, nylon column/tubes were eluted with 15 ul nuclease free water. The eluate was captured by spinning the tubes at 8,000×g for 2 minutes. A 1 μl portion of the eluate from each sample was loaded on a 1×TBE/8M urea, 15% polyacrylamide gel (Invitrogen). One Marker lane contained 0.5 ul RNA marker ladder, another contained 1 ul of the tRNA/RNA marker mixture used, and another contained 0.5 μl of bovine tRNA. Sample “SV 150 μl ethanol” was not run. Electrophoresis was performed at 120 volts (constant) for 2 hours at 21° C. The gel was removed from the plastic cassette and placed in a solution of 50 ml 1×TBE buffer plus a 5 μl aliquot of Sybr Gold (Invitrogen) and stained for 15 minutes with occasional mixing. The gel was digitally imaged using the Amersham Typhoon platform with settings of: 1. ex488/em526. 2. PMT 450.

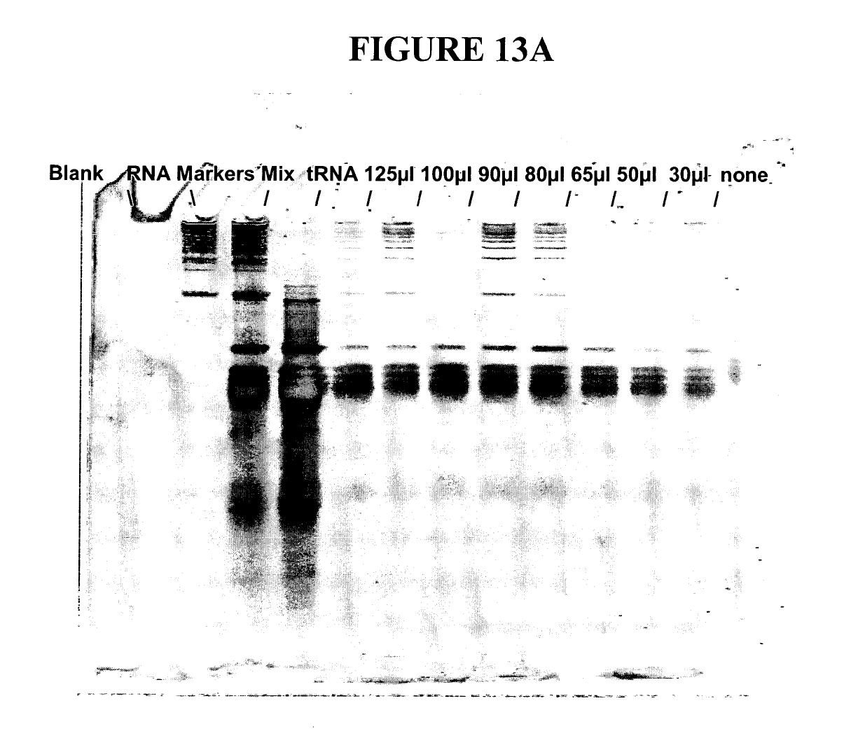

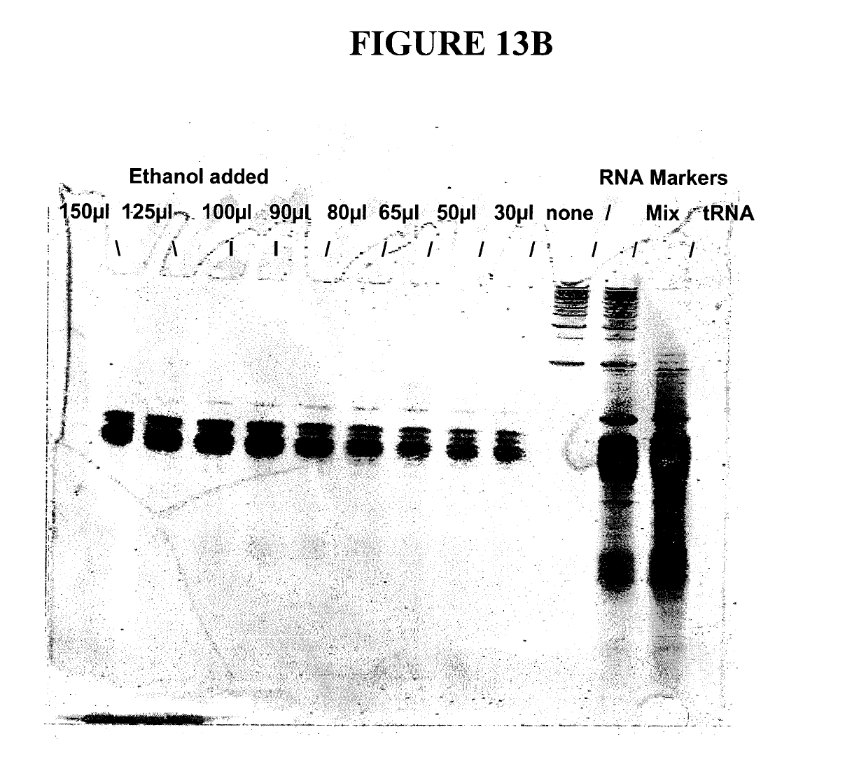

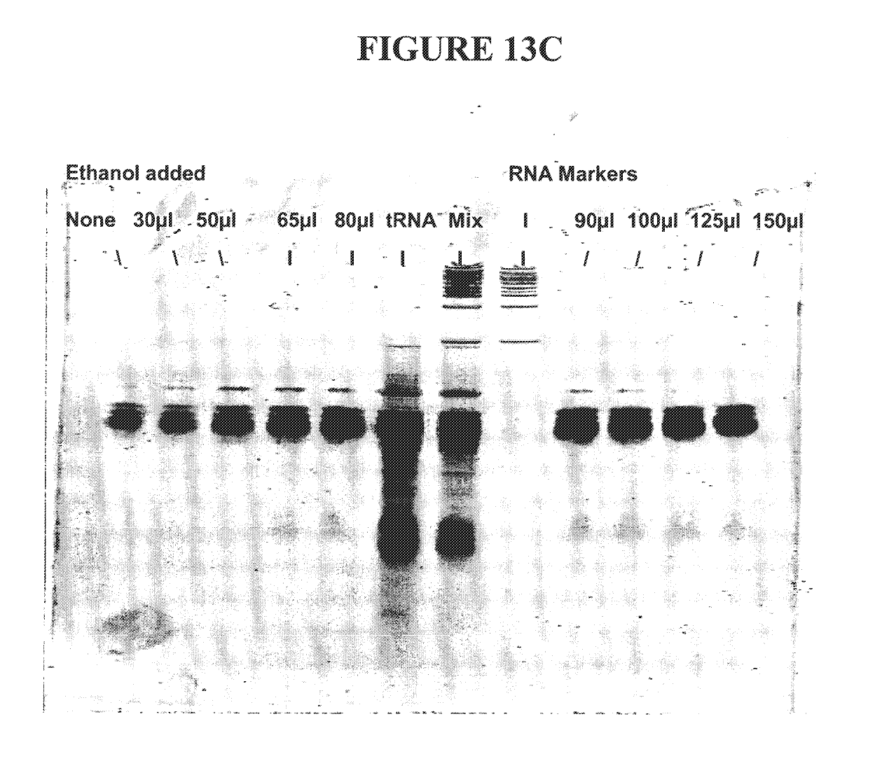

As shown in FIG. 13A (SV membranes), FIG. 13B (nylon membranes), and FIG. 13C (cellulose acetate membranes) this method was suitable for evaluating matrix materials for their utility as a binding matrix for this purification method. Further, the method also allowed for the approximate determination of the preferred amount of alcohol to be added, to provide preferred purification of small RNA molecules. Note that the use of alcohol was not required for the purification of small RNA molecules (for example, less than 200 bases) using SV, nylon or cellulose acetate matrices. For the use of SV (FIG. 13A), the addition of 30 μl or 50 μl of ethanol showed purification of small RNA but not of larger RNA molecules from the RNA Marker ladder. For nylon (FIG. 13B), the larger RNA molecules from the RNA Marker ladder were not visually purified for any ethanol amount added, with 90 μl added appearing about as good, or better, than any other amount added in terms of small RNA band intensities. For cellulose acetate (FIG. 13C), large RNA molecules from the RNA Marker ladder did not appear to purify using any amount of ethanol added, and the small RNA band intensities increased with increasing ethanol added. Using 90 or 100 μl ethanol added provided band intensities equivalent to or greater than any other ethanol addition amount. All three matrices showed binding conditions that allowed the preferential purification of small RNA molecules (e.g. those less than 200 bases) over larger RNA molecules (e.g. greater than 200 bases).

### Example 14

### Purification of Small RNA with No Chaotrope and Either No Alcohol or Various Concentrations of Alcohol

In this example, the purification of small RNA from a mixture of RNA using no chaotrope and either no alcohol or various concentrations of alcohol has been described. In a plastic tube, 20 μl of bovine tRNA 1 μg per ml (Promega part #Y209), 1400 of nuclease free water, and 40 μl RNA Markers (Promega cat #G3191) were combined. A 41 sample was removed for use as a gel marker (on two gels, see below). To this 198 μl mixture was added: (1) 75 μl 5M NaCl, (2) 300 μl 250 mM hexamminecobalt(III)chloride in water. No chaotrope was added. The tube was vortexed well and held at 21° C. for 5 minutes. Then 20 μl samples were placed into each of 8 tubes. To the eight tubes, 100% ethanol and nuclease free water were added so that the final (vol/vol) ethanol percentages were: 0%, 20%, 40%, 50%, 60%, 70%, 80%, 90% ethanol per tube, and the tubes were vortexed. 90 μl of each sample mixture was added to a nylon column (Corning Costar Spin-X catalog #8169) nested in a 1.5 ml tube. Then 90 μl of each sample mixture was added to a cellulose acetate column (Corning Costar Spin-X catalog #8160) nested in a 1.5 ml tube. All column/tubes were centrifuged at 8,000×g for 2 minutes. All nylon column/tubes and all cellulose acetate column/tubes were eluted with 20 ul nuclease free water. The eluate was captured by spinning the tubes at 8,000×g for 2 minutes. A 10 μl portion of the eluate from each sample was loaded on a 1×TBE/8M urea, 15% polyacrylamide gel (Invitrogen). One Marker lane contained 0.5 μl RNA marker ladder, another contained 1 μl of the tRNA/RNA Marker mixture used, and another contained 0.5 μl of bovine tRNA. Electrophoresis was performed at 120 volts (constant) for 2 hours at room temperature. The gel was removed from the plastic cassette and placed in a solution of 50 ml 1×TBE buffer plus a 50 aliquot of Sybr Gold (Invitrogen) and stained for 15 minutes with occasional mixing. The gel was digitally imaged using the Amersham Typhoon platform with settings of: 1. ex488/em526. 2. PMT 450.

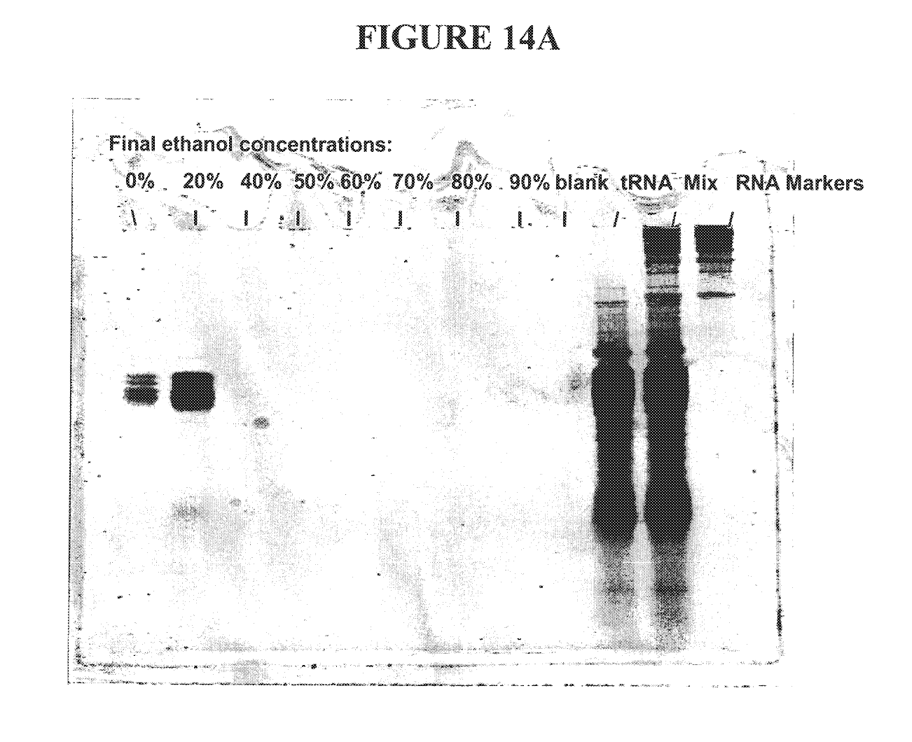

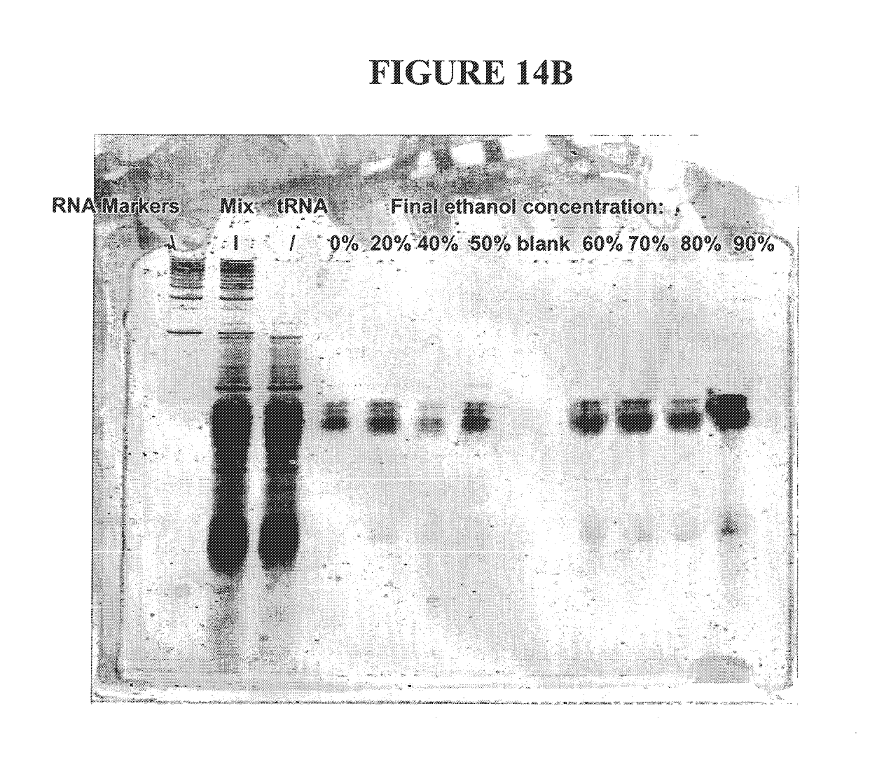

As shown in FIG. 14A (nylon membrane), and 14B (cellulose acetate membrane) this method was suitable for purifying small RNA molecules and evaluating matrix materials for their utility as a binding matrix. Further, the method also allowed for the approximate determination of the preferred amount of alcohol to be added to provide preferred purification of small RNA molecules. For nylon (FIG. 14A) or cellulose acetate (FIG. 14B), the larger RNA molecules from the RNA Marker ladder were not visually purified for any ethanol amount added. Both nylon and cellulose acetate matrices showed conditions that allowed the preferential purification of small RNA molecules (e.g. those less than 200 bases) over larger RNA molecules (e.g. greater than 200 bases).

### Example 15

### Small RNA Purification Using Different Ethanol Wash Concentrations

In this example, small RNA purification using either no wash step or a wash step with different ethanol concentrations has been described. In a plastic tube, 5 μl of bovine tRNA 1 μg per ml (Promega part #Y209), 30 μl of nuclease free water, and 30 μl RNA Markers (Promega cat #G3191) were combined. A 1 μl sample was removed for use as a gel marker (for use on three gel lanes, see below). To this 64 μl mixture was added: (1) 75 μl 5M NaCl, (2) 300 μl 250 mM hexamminecobalt(III)chloride in TE buffer and (3) 2.63 ml of 9M acetamide 115 mM TRIS pH7.5. The tube was vortexed well and held at 21° C. for 5 minutes. Then 2.0 ml of 95% ethanol was added, and the tube vortexed. 400 μl was added to each of 11 tubes. One tube was not washed with ethanol, the remaining 10 tubes were washed with 300 μl of 10%, 20%, 30%, 40%, 50%, 60%, 70%, 80%, 90%, and 100% ethanol. All column/tubes were centrifuged at 8,000×g for 2 minutes. All SV columns were removed to fresh tubes and each was eluted with 30 μl of nuclease free water, added directly to the membrane. The eluate was captured by spinning the tubes at 8,000×g for 2 minutes.

A 10 μl ortion of the eluate from each sample was loaded on a 1×TBE/8M urea, 15% polyacrylamide gel (Invitrogen). One Marker lane contained 1 ul of the tRNA/RNA marker mixture used. Electrophoresis was performed at 120 volts (constant) for 2 hours at 21° C. The gel was removed from the plastic cassette and placed in a solution of 50 ml 1×TBE buffer plus a 5 μl aliquot of Sybr Gold (Invitrogen) and stained for 15 minutes with occasional mixing. The gel was digitally imaged using the Amersham Typhoon platform with settings of: 1. ex488/em526. 2. PMT 450.

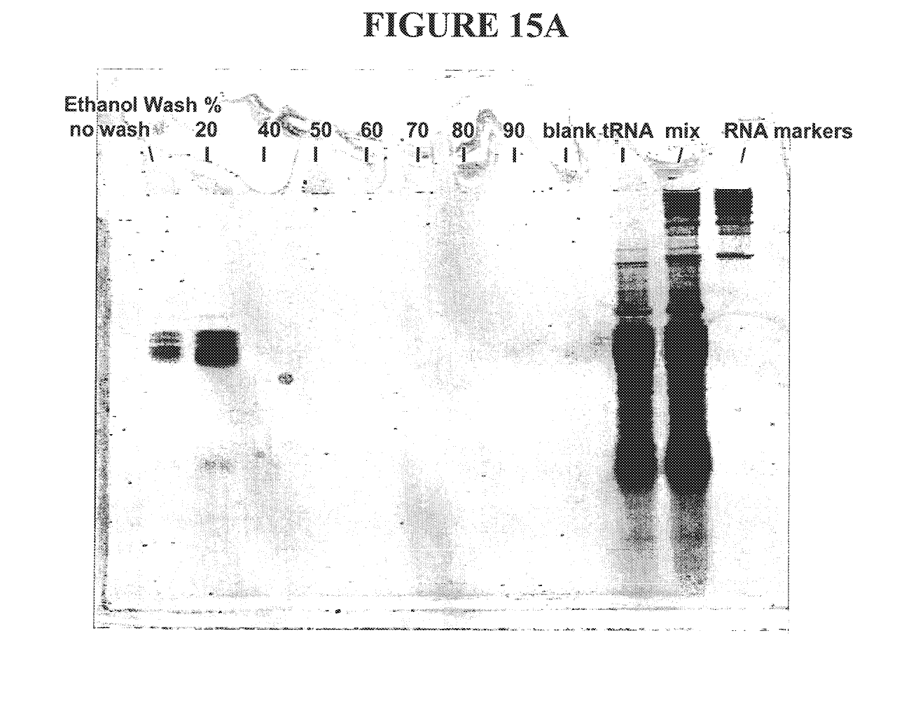

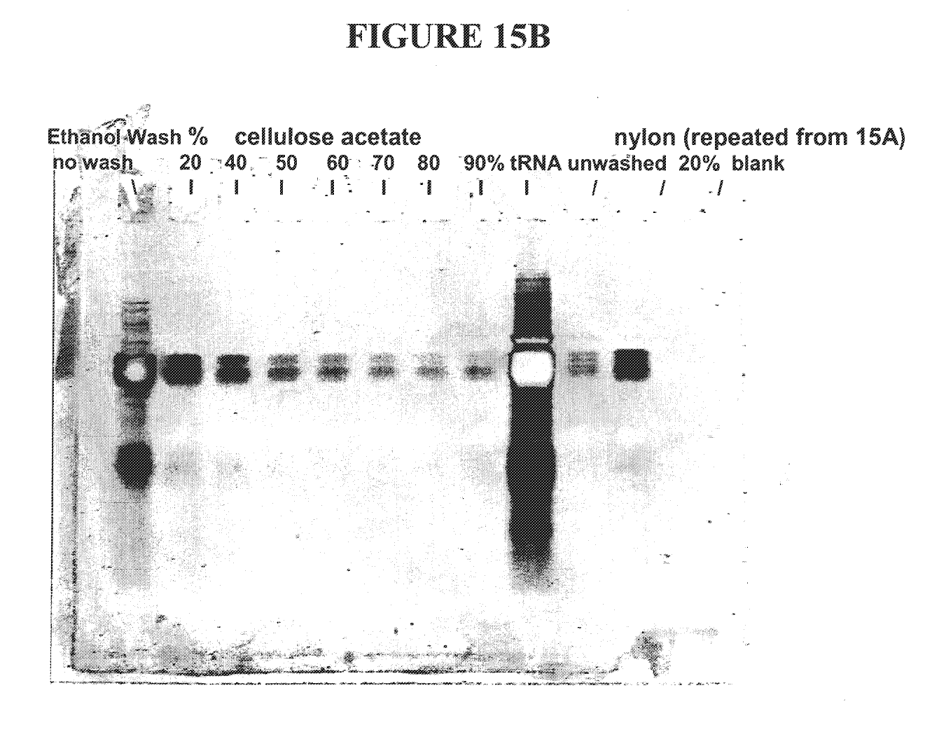

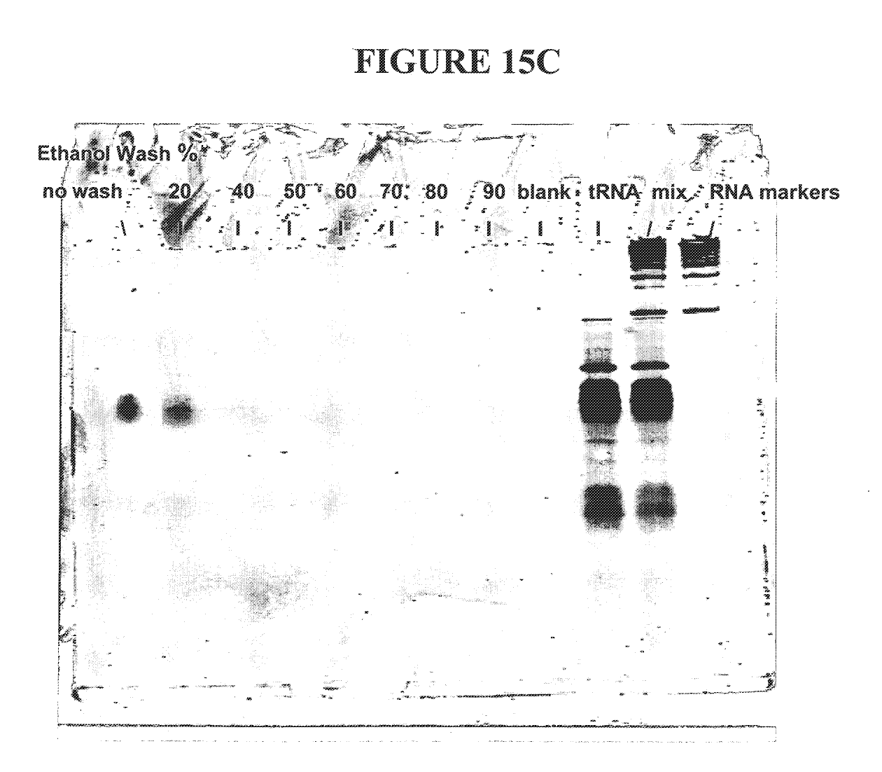

As shown in FIG. 15A (nylon membrane), the lower % ethanol washes retained RNA to various degrees, and 20% ethanol wash provided preferred purification than higher percentages such as 50% ethanol where visually no RNA was purified. As shown in FIG. 15B (cellulose acetate membrane) 20% and 40% ethanol washes provided preferable purification than higher percentages, such as 70% or 80% ethanol washes. As shown in FIG. 15C (SV membranes) the 20% ethanol wash was preferable. This is surprising and quite different from ethanol washes in various commercial kits where about 80% ethanol is routinely used.

### Example 16

### Small RNA Purification with Paramagnetic Particles and Various Chaotropes

In this example, small RNA purification with magnetic particles and various chaotropes has been described. To each of 9 tubes was added 5 μl, 10 μl or 15 μl (in duplicate) of MagneSil® Blue paramagnetic particles (Promega cat #A220). The tubes were placed on a supermagnet and held for 30 seconds. The supernatant was removed. The MagneSil® Blue was rinsed twice with 500 μl aliquots of either: (a) 1.8M urea, 20 mM TRIS pH7.5, 125 mM NaCl, 25 mM Hexamminecobalt(III)chloride (b) 9M acetamide, 115 mM TRIS pH7.5, 125 mM NaCl, 25 mM Hexamminecobalt(III)chloride or (c) 2M thiourea, 20 mM TRIS pH7.5, 125 mM NaCl, 25 mM Hexamminecobalt(III)chloride.

With each rinse the MagneSil® Blue was resuspended and held for 5 minutes at 21° C. The tube was placed on a supermagnet and held for 30 seconds. The supernatant was then removed. 1×10⁶ cultured 293T human cells were washed twice with 200 μl 1×PBS pH6.8. PBS supernatant was removed after centrifugation of cells. A 2000 aliquot of either (a) 1.8M urea, 20 mM TRIS pH7.5, 125 mM NaCl, 25 mM Hexamminecobalt(III) chloride, (b) 9M acetamide, 115 mM TRIS pH7.5, 125 mM NaCl, 25 mM Hexamminecobalt(III) chloride or (c) 2M thiourea, 20 mM TRIS pH7.5, 125 mM NaCl, 25 mM Hexamminecobalt(III)chloride was added to each of three separate tubes containing washed cultured cells. The tubes were vortexed and held for 5 minutes at 21° C. The cell lysate mixture was then transferred to each of three tubes containing either 5 μl, 10 μl or 15 μl of previously rinsed MagneSil® Blue. A 530 μl aliquot of 75% ethanol was added to each tube and the MagneSil® Blue was resuspended. After a 5 minute hold at 21° C. with mixing every 30 seconds the tubes were placed on a supermagnet for 30 seconds. The supernatant was removed. The MagneSil® Blue was rinsed twice with 500 μl aliquots of 75% ethanol. After removing the final rinse, the Magnesil® Blue was allowed to dry on the supermagnet for 10 minutes. A 50 μl aliquot of nanopure water was added to each tube containing MagneSil® Blue and held at 21° C. for 5 minutes. The eluate was removed after placing the MagneSil® Blue on a supermagnet. A 5 μl portion of the eluate from each tube was mixed with 5 μl of 2× formamide loading dye (Ambion) and heated at 80° C. for 3 minutes. This mixture was then loaded on a 1×TBE/8M urea, 15% polyacrylamide gel (Invitrogen). One Marker lane contained 100-500b markers (Ambion). An additional marker lane (25, 45 and 70b) contained small T7 runoff transcripts produced from cut plasmids (pGEM-3zf+, and pGEM-5zf+). Electrophoresis was performed at 100 volts (constant) for 2 hours at 21 C. The gel was removed from the plastic cassette and placed in a solution of 50 ml 1×TBE buffer plus a 5 μl aliquot of Sybr Gold (Invitrogen) and stained for 5 minutes with occasional mixing. The gel was digitally imaged using the Amersham Typhoon platform with settings of: 1. ex488/em526. 2. PMT 450.

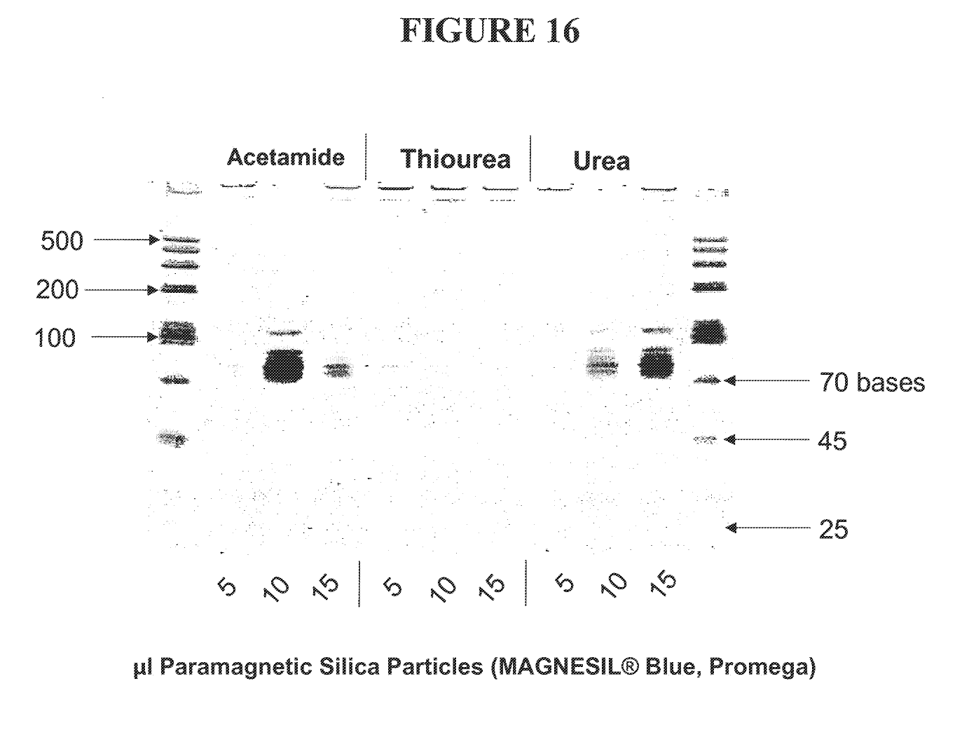

As shown in FIG. 16, a magnetic silica matrix such as Magnesil® Blue can be used to purify small RNA's from cultured cells.

### Example 17

### Small RNA Purification Using SV96 Binding Plates and Various Chaotropes

In this example, the purification of small RNA using SV96 binding plates and various chaotropes has been described. 1×106 cultured 293T human cells were washed twice with 200 μl 1×PBS pH6.8 to remove cell culture media. PBS supernatant was removed after centrifugation of cells. A 200 ul aliquot of either 2M thiourea 20 mM TRIS pH7.5 125 mM NaCl 25 mM Hexamminecobalt(III)chloride, 9M acetatamide 115 mM TRIS pH7.5 125 mM NaCl 25 mM Hexamminecobalt(III)chloride, or 8M urea 20 mM TRIS pH7.5 125 mM NaCl 25 mM Hexamminecobalt(III)chloride was added to the each of 2 separate tubes. Tubes were vortexed and held at 21° C. for 5 minutes. Then 530 μl of 75% ethanol was added to each tube, vortexed and held at 21° C. for 5 minutes. Each lysate with differing chaotropes was added directly to individual wells of a SV96 plate (Promega cat #A227). Vacuum was applied to draw lysate through the membranes. SV96 well membranes were washed twice with separate 500 μl aliquots of 75% ethanol with vacuum applied. The plate was held under vacuum for 5 minutes after the last rinse to dry the membranes. An 80 μl aliquot of nanopure water was added directly to the membrane of each well and held at 21° C. for 5 minutes. The eluate was captured in a 96 well polypropylene plate under vacuum. A 5 μl portion of the eluate from each column was mixed with 5 μl of 2× formamide loading dye (Ambion) and heated at 80° C. for 3 minutes.

This mixture was then loaded on a 1×TBE/8M urea, 15% polyacrylamide gel (Invitrogen). One Marker lane contained 100-500b markers (Ambion). An additional marker lane (25, 45 and 70b) contained small T7 runoff transcripts produced from cut plasmids (pGEM-3zf+, and pGEM-5zf+). Electrophoresis was performed at 100 volts (constant) for 2 hours at room temperature. The gel was removed from the plastic cassette and placed in a solution of 50 ml 1×TBE buffer plus a 50 aliquot of Sybr Gold (Invitrogen) and stained for 5 minutes with occasional mixing. The gel was digitally imaged using the Amersham Typhoon platform with settings of: 1. ex488/em526. 2. PMT 450.

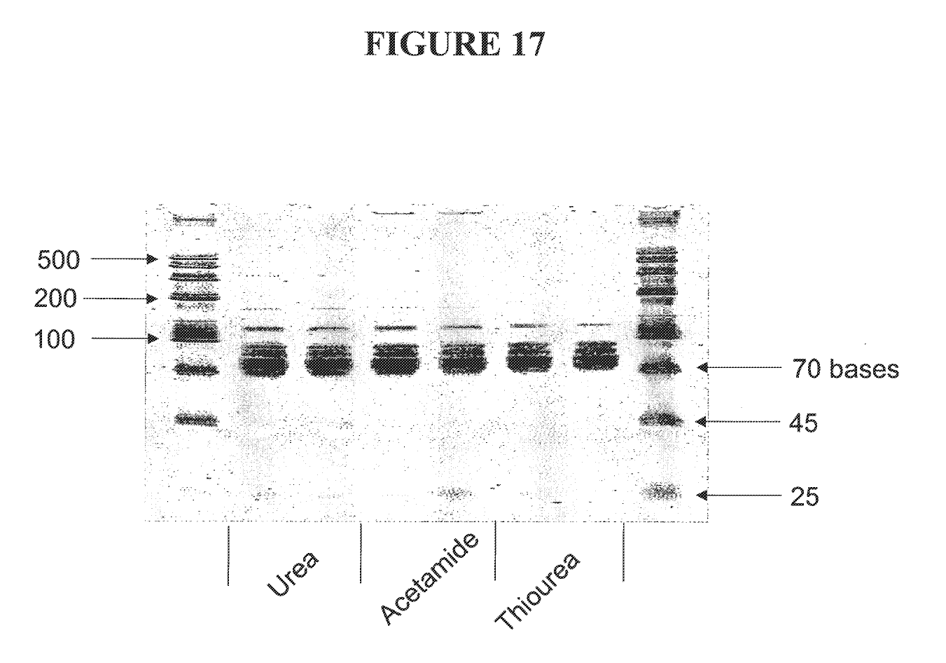

As shown in FIG. 17, 96 well plates with silica matrix such as SV96 can be used to purify small RNAs from cultured cells.

### Example 18

### Small RNA Purification with Hexaminenickel (II)

In this example, the purification of small RNA using hexamminenickel (II) and acetamide has been described. 5.0 g of nickel (II) chloride hexahydrate (Aldrich cat #223387-500G) was dissolved in 10 ml nanopure water in a 250 ml glass beaker. 40 ml of ice cold aqueous 14.8 N ammonium hydroxide (Fisher cat #A669-212) was added. This mixture was placed on ice for 30 minutes with occasional mixing. Hexamminenickel (II) chloride crystals were captured using a Whatman #4 filter paper disc in a Buchner funnel using vacuum filtration. The crystals were washed once with 10 ml of ice cold aqueous ammonium hydroxide. Crystals were then rinsed with four separate volumes of 25 ml 95% ethanol. After the final rinse the crystals on the filter paper were held under vacuum for 5 minutes.

60 mg of synthesized Hexamminenickel (II) chloride crystals was placed in a microfuge tube. 675 μl of nanopure water was added, followed by 125 μl of 2 M TRIS pH 7.5, and 200 μl 6 N HCl. To each of 7 tubes was added 1×10⁶ cultured 293T human cells previously washed twice with 200 μl 1×PBS pH 6.8. PBS supernatant was removed after centrifugation of cells. 175 μl of 9 M acetamide, 115 mM TRIS pH 7.5 was added to each of seven separate tubes containing washed cultured cells. 4 μl of 5 M NaCl was added to each tube followed by addition of 0 μl, 1 μl, 2.5 μl, 5 μl, 10 μl, 15 μl, or 20 μl of Hexamminenickel (II) chloride solution to each of seven separate tubes. Additional 9 M acetamide, 115 mM TRIS pH 7.5 was added to each tube so that the final volume was 200 μl. The tubes were vortexed and held for 5 minutes at 21° C. 530 μl of 75% ethanol was added to each tube, vortexed and held at 21° C. for 5 minutes. Each lysate with differing concentrations of Hexamminenickel (II) chloride was added directly to each of seven separate SV spin columns (Promega cat #Z3111) and centrifuged at 2,000×g for 2 minutes.

Each column membrane was rinsed with 500 μl of 40% ethanol (v/v with nanopure water). Each SV column was centrifuged a final time at 8,000×g for 5 minutes. 35 μl of nanopure water was added directly to the membrane of each column and held at 21° C. for 5 minutes. The eluate was captured in a new microfuge tube by centrifugation at 8,000×g for 2 minutes.

A 5 μl portion of the eluate from each column was mixed with 5 μl of 2× formamide loading dye (Ambion) and heated at 80° C. for 3 minutes. This mixture was then loaded on a 1×TBE/8M urea, 15% polyacrylamide gel (Invitrogen). One Marker lane contained 100-500b markers (Ambion). An additional marker lane (25, 45 and 70b) contained small T7 runoff transcripts produced from cut plasmids (pGEM-3zf+, and pGEM-5zf+).

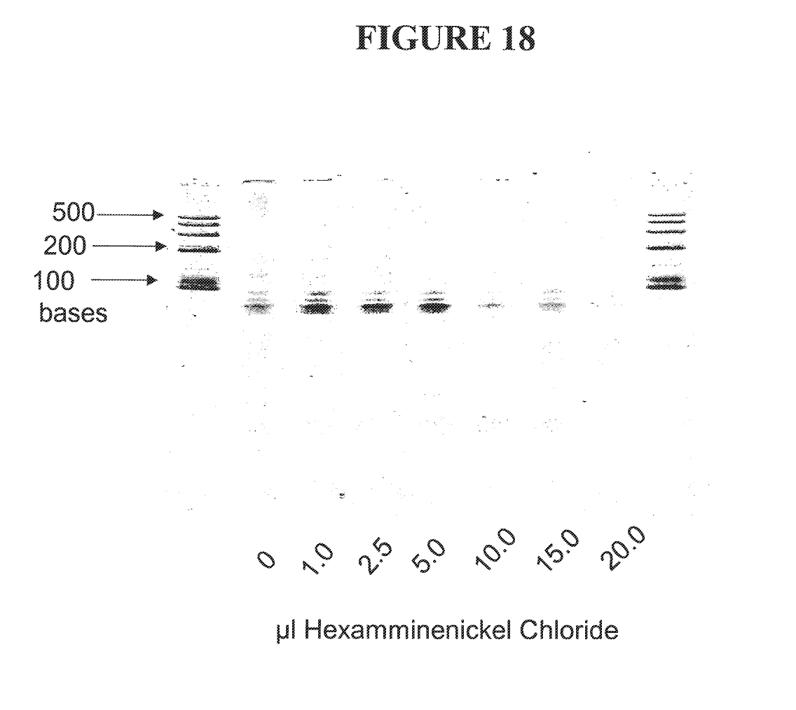

Electrophoresis was performed at 125 volts (constant) for 2 hours at 21° C. The gel was removed from the plastic cassette and placed in a solution of 50 ml 1×TBE buffer plus 5 μl of Sybr Gold (Invitrogen) and stained for 5 minutes with occasional mixing. The gel was digitally imaged using the Amersham Typhoon platform with settings of: 1. ex488/em526. 2. PMT 450. As shown in FIG. 18, hexamminenickel (II) chloride can be synthesized and used to isolate small RNA's from cultured human cells.

### Example 19

### Effect of Various Percent Ethanol Rinses

This Example describes the purification of small RNA using various percentages of ethanol for rinsing the RNA bound membrane. 1×10⁶ cultured 293T human cells were washed twice with 200 μl 1×PBS pH 6.8 to remove cell culture media. PBS supernatant was removed after centrifugation of cells. 200 μl of 9M acetamide, 115 mM TRIS pH7.5, 125 mM NaCl, 25 mM Hexamminecobalt(III)chloride was added to the each of nine separate tubes. Tubes were vortexed and held 21° C. for 5 minutes. 530 μl of 75% ethanol was added to each tube, vortexed and held at 21° C. for 5 minutes. Each lysate was added directly to each of nine separate SV columns and centrifuged at 2,000×g for 2 minutes. Each column membrane was rinsed with 500 μl of either 0%, 20%, 30%, 40%, 50%, 60%, 70%, 80%, or 90% ethanol (v/v with nanopure water). Each SV column was centrifuged a final time at 8,000×g for 5 minutes. 35 μl of nanopure water was added directly to the membrane of each column and held at 21° C. for 5 minutes. The eluate was captured in a new microfuge tube by centrifugation at 8,000×g for 2 minutes.

A 5 μl portion of the eluate from each column was mixed with 5 μl of 2× formamide loading dye (Ambion) and heated at 80° C. for 3 minutes. This mixture was then loaded on a 1×TBE/8M urea, 15% polyacrylamide gel (Invitrogen). One Marker lane contained 100-500b markers (Ambion). An additional marker lane (25, 45 and 70b) contained small T7 runoff transcripts produced from cut plasmids (pGEM-3zf+, and pGEM-5zf+).

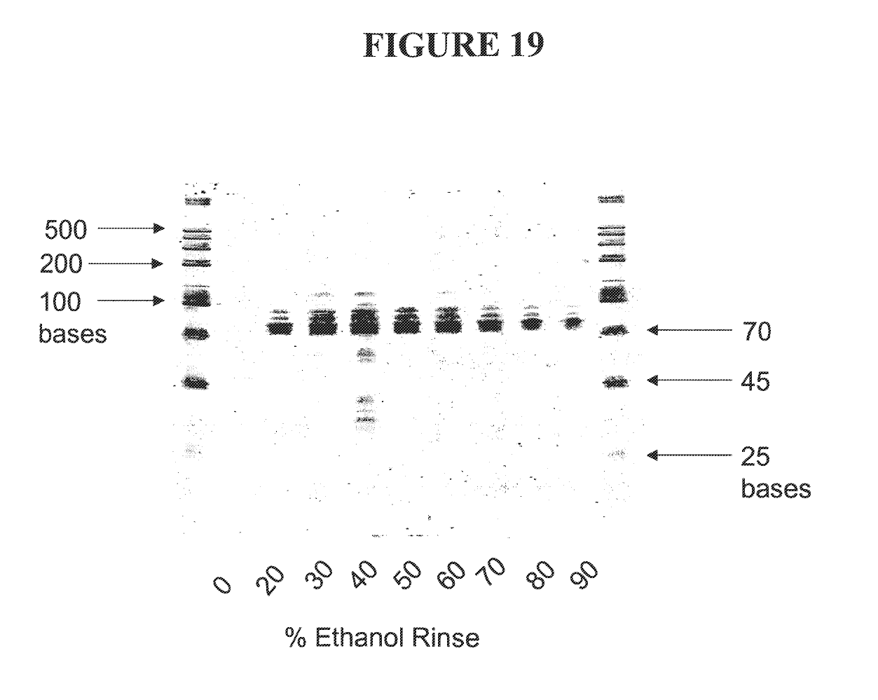

Electrophoresis was performed at 125 volts (constant) for 2 hours at 21° C. The gel was removed from the plastic cassette and placed in a solution of 50 ml 1×TBE buffer plus 5 μl of Sybr Gold (Invitrogen) and stained for 5 minutes with occasional mixing. The gel was digitally imaged using the Amersham Typhoon platform with settings of: 1. ex488/em526. 2. PMT 450. As shown in FIG. 19, a greater amount small RNA's can be isolated when using SV membranes if rinsed with 30%-50% ethanol.

### Example 20

### Small RNA Purification Using Ruthenium Hexamine Chloride

This Example describes the purification of small RNA using ruthenium hexamine trichloride and acetamide. A 250 mM ruthenium hexammine trichloride (Polysciences Inc., Warrington, Pa., cat #17253-1) solution was prepared with 1×TE pH 8.0. To each of seven tubes was added 1×10⁶ cultured 293T human cells previously washed twice with 200 μl 1×PBS pH 6.8. PBS supernatant was removed after centrifugation of cells. 175 μl of 9 M acetamide, 115 mM TRIS pH7.5 was added to each of seven separate tubes containing washed cultured cells. 5 μl of 5 M NaCl was added to each tube followed by addition of 0 μl, 1 μl, 2.5 μl, 5 μl, 10 μl, 15 μl or 20 μl of ruthenium hexammine trichloride solution to each separate tube. Additional 9 M acetamide, 115 mM TRIS pH 7.5 was added to each tube so that the final volume was 200 μl. The tubes were vortexed and held for 5 minutes at 21° C. 530 μl of 75% ethanol was added to each tube, vortexed and held at 21° C. for 5 minutes. Each lysate with differing concentrations of ruthenium hexammine trichloride was added directly to each of seven separate SV spin columns (Promega cat #Z3111) and centrifuged at 2,000×g for 2 minutes.

Each column membrane was rinsed once with 500 μl of 40% ethanol (v/v with nanopure water). Each SV column was centrifuged a final time at 8,000×g for 5 minutes. A 35 μl sample of nanopure water was added directly to the membrane of each column and held at 21° C. for 5 minutes. The eluate was captured in a new microfuge tube by centrifugation at 8,000×g for 2 minutes.

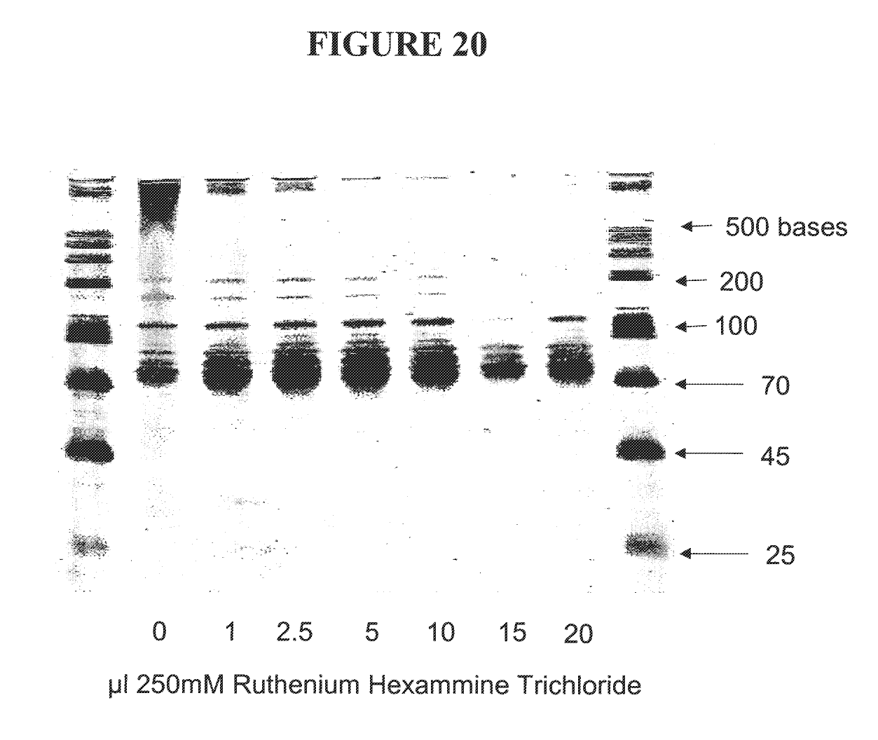

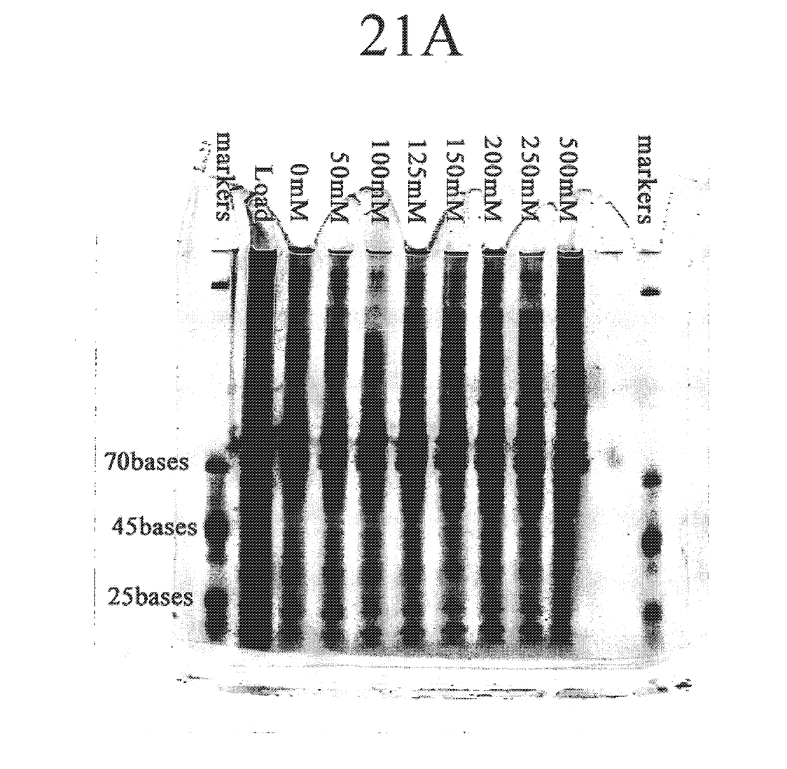

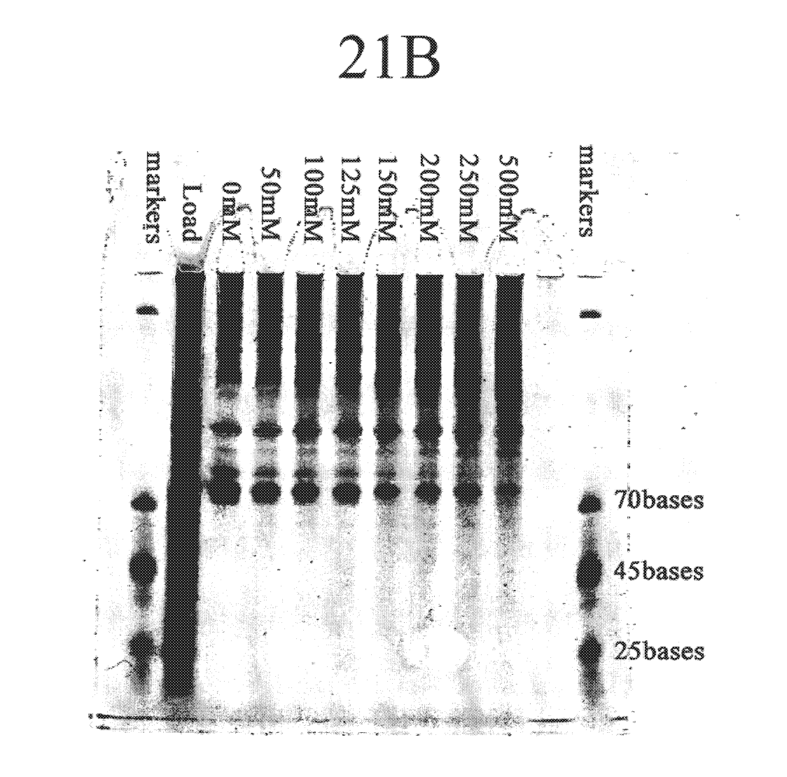

A 5 μl portion of the eluate from each column was mixed with 5 μl of 2× formamide loading dye (Ambion) and heated at 80° C. for 3 minutes. This mixture was then loaded on a 1×TBE/8M urea, 15% polyacrylamide gel (Invitrogen). The marker lane contained 100-500b markers (Ambion). Electrophoresis was performed at 125 volts (constant) for 2 hours at 21° C. The gel was removed from the plastic cassette and placed in a solution of 50 ml 1×TBE buffer plus 5 μl of Sybr Gold (Invitrogen) and stained for 5 minutes with occasional mixing. The gel was digitally imaged using the Amersham Typhoon platform with settings of: 1. ex488/em526. 2. PMT 450. As shown in FIG. 20, ruthenium hexamine trichloride can be used to isolate small RNA's from cultured human cells.

### Example 21

### Making Nickel Hexamethylammine Chloride

This example describes the method used to generate nickel hexamethylammine chloride. Two (2.0) gm of NaOH was dissolved in 10 ml of water. 3.0 gm of methylamine hydrochloride (Sigma cat #M0505) was dissolved into this solution. Total volume was about 10.5 ml. 15 ml of isopropanol was added and mixed. A white crystalline precipitate was formed, resembling NaCl. The solution became biphasic with about 8 ml of lower phase and about 15 ml of upper phase. 8.0 ml of the lower phase was pipetted out to a fresh tube, with care taken not to remove the white precipitate. 1.0 gm of nickel chloride (Sigma cat #223387) was added to 2 ml of water, and mixed until dissolved. This solution was added to the above 8 ml solution in a 50 ml plastic tube, and mixed. A green precipitate was formed: nickel hexamethylammine chloride. The solution was placed into a Buchner funnel containing a sheet of Whatman #4 filter paper, and the contents vacuum filtered, leaving the pale green precipitate. This was washed 3 times with 10 ml of water per wash. The precipitate was then removed to a 50 ml plastic tube and air dried overnight to remove water.

### Example 22

### Making Nickel Hexaethylammine Chloride

This examples describes the procedure used to make nickel hexaethylammine chloride. 2.0 gm of NiCl was added to 7.5 ml of water, and mixed until dissolved, and then pipetted into 5.0 gm of 70% ethylamine solution (Sigma cat #E3754) in a 50 ml plastic tube, and mixed. A deep green precipitate formed: nickel hexaethylammine chloride. This was washed and air dried as described in Example 21 for nickel hexamethylammine.

### Example 23

### Coating a Silica Surface with Nickel Hexamethylammine Chloride

This example describes a procedure used to coat a silica surface with nickel hexamethylammine chloride. 2.0 gm of NaOH was dissolved in 10 ml of water. 3.0 gm of methylamine hydrochloride (Sigma cat #M0505) was dissolved into this solution. Total volume was about 10.5 ml. 15 ml of isopropanol was added and mixed. A white crystalline precipitated was formed, resembling NaCl. The solution became biphasic with about 8 ml of lower phase and about 15 ml of upper phase. 8.0 ml of the lower phase was pipetted out to a fresh tube, with care taken not to remove the white precipitate. 1.0 gm of nickel chloride (Sigma cat #223387) was added to 2 ml of water, and mixed until dissolved. This solution was added to the above 8 ml solution in Pyrex® glass beaker, and mixed. A green precipitate formed a coating (nickel hexamethylammine chloride) on the silica surface of the beaker. This coating was washed with sterilized nanopure water, and dried.

### Example 24

### Coating a Silica Surface with Nickel Hexaethylammine Chloride

This example describes a procedure used to coat a silica surface with nickel hexaethylammine chloride. 2.0 gm of NiCl was added to 7.5 ml of water, and mixed until dissolved, and then pipetted into 5.0 gm of 70% ethylamine solution (Sigma cat #E3754) in a Pyrex® glass beaker, and mixed. A deep green precipitate formed: nickel hexaethylammine chloride. This was washed and dried as described in Example 23 for silica coated with nickel hexamethylammine. The surface coating of the transition metal complexes was not removed during serial washes with water, because the transition metal complex was low in water solubility. Washing the surfaces generated in examples 23 and 24 with solutions containing imidazole or histidine allowed the removal of the transition metal complexes from the silica surfaces.

### Example 25

### Binding and Elution of RNA Bound to Columns Pretreated with Hexammine Cobalt Chloride

In this example, total RNA was bound to SV columns treated prior to use by applying 35 μl of 250 mM hexammine cobalt chloride and allowing it to enter by absorption. This 35 μl volume was representative of the dead volume of the column matrix. Furthermore, the addition of defined molarities of NaCl to cell lysates allowed a size selectivity to be attained whereby smaller RNAs were excluded from the binding matrix.

175 μl of 7.4M acetamide, 1.4% NP-9, 2% beta-mercaptoethanol, pH 4.8 was added to cell pellets representing 1×10⁶ HeLa cells in eight individual 1.5 ml Eppendorf tubes, resuspended by vortexing. After five minutes static incubation at 21° C., 25 μl of prepared aqueous NaCl solutions at 0.0, 0.4, 0.8, 1.0, 1.2, 1.6, 2.0, or 4.0M were added respectively to eight sample tubes, and mixed by vortexing. The resultant mixes were at final NaCl concentrations of 0, 50, 100, 125, 150, 200, 250, and 500 mM. Columns pretreated with hexammine cobalt chloride were used for separations. Each 200 μl sample was pipetted onto a single column followed by 20 seconds centrifugation at 12,000×g. Flow through fluids were collected and set aside for later analysis (see FIG. 21A). 500 μl of column wash solution composed of 5 mM EDTA, 65% EtOH, pH 8 was applied to each column followed by 20 seconds centrifugation at 12,000×g. Flowthroughs were discarded and the wash procedure repeated once. A third centrifugation at 12,000×g for two minutes was done to eliminate residual EtOH and dry the columns. Finally, 50 μl of 10 mM Tris, 0.1 mM EDTA, pH 8 was applied to each column to elute bound RNA. The eluted RNA was collected by 60 second centrifugation at 12,000×g (see FIG. 21B).

Electrophoretic analysis using 10% of each purified RNA or saved column flow through was performed using 15% acrylamide/6M urea gels applying 125V for 2 hours. Gels were stained using a 1:10,000 dilution of SYBR Gold (Invitrogen cat #S11494) for five minutes at room temperature. Results were obtained by digital imaging using the Amersham Typhoon scanner and settings of excitation 488 nm/emission 526 nm and a PMT of 450.

A visible increase in exclusion of small RNA from binding to the hexammine cobalt chloride pretreated SV columns was observed as the molarity of NaCl was increased across the sample series.

### Example 26

### Making Cobalt Hexamethylammine Chloride

This examples describes a method used to make cobalt hexamethylammine chloride. 4.0 gm of NaOH was dissolved in 20 ml of water. 6.0 gm of methylamine hydrochloride (Sigma cat #M0505) was dissolved into this solution. 15 ml of isopropanol was added and mixed. A white crystalline precipitate was formed, resembling NaCl. The solution became biphasic with an aqueous lower phase (containing methylamine hydroxide) and an upper phase containing isopropanol. The lower phase was pipetted out to a fresh tube, with care taken not to remove the white precipitate. 2.37 gm of cobalt chloride (Sigma cat #C8661, 1/10^{th} mole) was added to 20 ml of water, and mixed until dissolved. This solution was added to the methylamine hydroxide solution and mixed. A dark green precipitate was formed: cobalt hexamethylammine chloride. The solution was placed into a Buchner funnel containing a sheet of Whatman #4 filter paper, and the contents vacuum filtered, leaving the red precipitate. This was washed 4 times with 20 ml of water per wash. The precipitate was then removed to a 50 ml plastic tube and air dried overnight to remove water. The next morning, the tube was scraped with a metal spatula to produce a powder, and this was vacuum dried at 21° C. for 2 hours.

### Example 27

### Making Cobalt Hexaethylammine Chloride

This example describes a method used to make cobalt hexaethylammine chloride. 2.37 gm of CoCl hexahydrate (Sigma cat #C8661, 1/10^{th} mole) was added to 20 ml of water in a 50 ml plastic screw cap tube, and mixed until dissolved, and then pipetted into 3.2 gm of 70% ethylamine solution (Sigma cat #E3754), and mixed. A light green precipitate formed: cobalt hexaethylammine chloride. This was washed, air dried and vacuum dried as described in Example 26 for cobalt hexamethylammine.

### Example 28

### Making Various Compaction Agents

This example describes the methods used to make various compaction agents, including: cobalt monoethanolammine pentaethylammine chloride, cobalt diethanolamminetetraethylammine chloride, cobalt monoethanolammine pentaethylammine sulfate, and cobalt diethanolamminetetraethylammine sulfate. 2.38 gm of cobalt chloride (CoCl hexahydrate (Sigma cat #C8661, 1/10^{th} mole)) was added to 20 ml of 9.0M acetamide/25 mM NaOAc, pH 5.2 in a 50 ml plastic tube, and 10.15 gm of Cobalt Sulfate, 7×H₂O (Sigma #C6768) was added to 30 ml of 9.0M acetamide/25 mM NaOAc, pH 5.2 in a 50 ml plastic tube, and each was mixed until dissolved. Two solutions were made in 50 ml plastic tubes: (a) “monoethanolammine pentaethylammine”: 0.61 gm of ethanolamine (Sigma #E9508) was added to 3.86 gm of ethylamine, 70% (Sigma #E3754) and mixed thoroughly, and solution (b) “diethanolamminetetraethylammine”: 1.22 gm of ethanolamine was added to 2.57 gm of ethylamine, 70%, and mixed thoroughly. Both solution (a) and (b) were each divided into two equal volumes in 50 ml plastic tubes.

10 ml of the CoCl solution (above) was added to the first solution of (a), a deep green precipitate was formed. The precipitant was a mixture of cobalt hexaethylamine chloride, cobalt monoethanolammine pentaethylammine chloride, cobalt diethanolamminetetraethylammine chloride, triethanolammine triethylammine chloride, cobalt tetraethanolamminediethylammine chloride, pentaethanolammine diethylammine chloride, cobalt hexaethanolammine chloride. Transition metal complexes with higher content of ethanolamine (relative to ethylammine) showed a higher solubility. To reduce the contribution of the more soluble components of the mixture, the precipitant was washed twice with 10 ml of 9.0M acetamide/25 mM NaOAc, pH 5.2. The less soluble transition metal complexes with lower content of ethanolamine (relative to ethylammine) tended to remain in a precipitated form. Thus after two washes, cobalt monoethanolammine pentaethylammine chloride was the principal transition metal complex formed, based on spectrophotometric scans.

The remaining 10 ml of CoCl (above) was added to the first solution (b), and a deep green precipitate was formed which was a mixture of transition metal complexes as described above. To reduce the contribution of the more soluble components of the mixture, the precipitant was washed twice with 10 ml of 9.0M acetamide/25 mM NaOAc, pH 5.2. After this series of washes, cobalt diethanolammine tetraethylammine chloride was the principal transition metal complex in solution, based on spectrophotometric scans.

15 ml of the above cobalt sulfate solution was added to the second solution of (a), and a deep green precipitate was formed, as described above for the transition metal chloride salts. The sulfate salts in 9.0M acetamide/25 mM NaOAc, pH 5.2, were less soluble than the corresponding chloride salts. After the precipitant was washed twice with 10 ml of 9.0M acetamide/25 mM NaOAc, pH 5.2, as described above, the principal transition metal complex in solution, based on spectrophotometric scans, was cobalt monoethanolammine pentaethylammine sulfate.

The remaining 15 ml of the above cobalt sulfate solution was added to the second solution of (b), and a deep green precipitate was formed, as described above for the transition metal chloride salts. The sulfate salts in 9.0M acetamide/25 mM NaOAc, pH 5.2, were less soluble than the corresponding chloride salts. After the precipitant was washed with 10 ml of 9.0M acetamide/25 mM NaOAc, pH 5.2, as described above, the principal transition metal complex in solution, based on spectrophotometric scans, was cobalt diethanolammine tetraethylammine sulfate.

### Example 29

### Methods of Screening the Binding of Transition Metal Complexes to a Mixture of Oligonucleotides

This example describes methods used to screen various transition metal complexes against a mixture of RNA and DNA oligonucleotides. The mixture of RNA and DNA oligonucleotides was made by combining the following:

(21 bases, SEQ ID NO: 1)
RNA₁ =
5′-UAUUGCACUUGUCCCGGCCUG-3′;
(25 bases, SEQ ID NO: 2)
RNA₂ =
5′-GAGACCCAGUAGCCAGAUGUAGCUU-3′;
(SEQ ID NO: 3)
RNA_{2-COMPL}(“RNA_{2′}”) =
5′-AAGCUACAUCUGGCUACUGGGUCUC-3′ 25b;
(35 bp, SEQ ID NO: 4)
DNA_{A} =
5′-AGCTGTCTAGGTGACACGCTAGAGTACTCGAGCTA-3′;
(SEQ ID NO: 5)
DN_{A′-COMPL} =
5′-TAGCTCGAGTACTCTAGCGTGTCACCTAGACAGCT-3′;
(30 bases, SEQ ID NO: 6)
DNA_{B} =
5′-GTTACACATGCCTACACGCTCCATCATAGG-3′.

There was an excess of either one of the complementary sequences (for example RNA₂ and its complementary sequence, denoted as “RNA_{2-COMPL}” or “RNA_{2′},”) or the other oligonucleotide, so that one of the single stranded RNA (25 base), or DNA (35 base) oligonucleotides was present in the mixture, in addition to the double stranded DNA or double stranded RNA which was composed of the two hybridized complementary sequences.

1.5 ul of the above oligonucleotide mix was added to 10 ul of the transition metal complex in 9.0M acetamide/25 mM NaOAc, pH 5.2, mixed and incubated at 21° C. for 2 minutes. Then 1.5 ul of MagneSil® (Promega catalog A2201) paramagnetic silica particles were added, mixed and incubated at 21° C. for 20 minutes. The sample mixtures were magnetized for 2 minutes, and the supernatant fractions removed to clean tubes. For each sample, 8 ul of the supernatant was added to 5 ul of 6× blue/orange loading dye (Promega catalog G1881), and the sample loaded into the well of the gel described below. To the magnetic particles from each sample, 10 ul of nuclease free water was added, and 5 ul of 6× dye was added, the solution mixed and loaded (including particles) into the well of the gel described below.

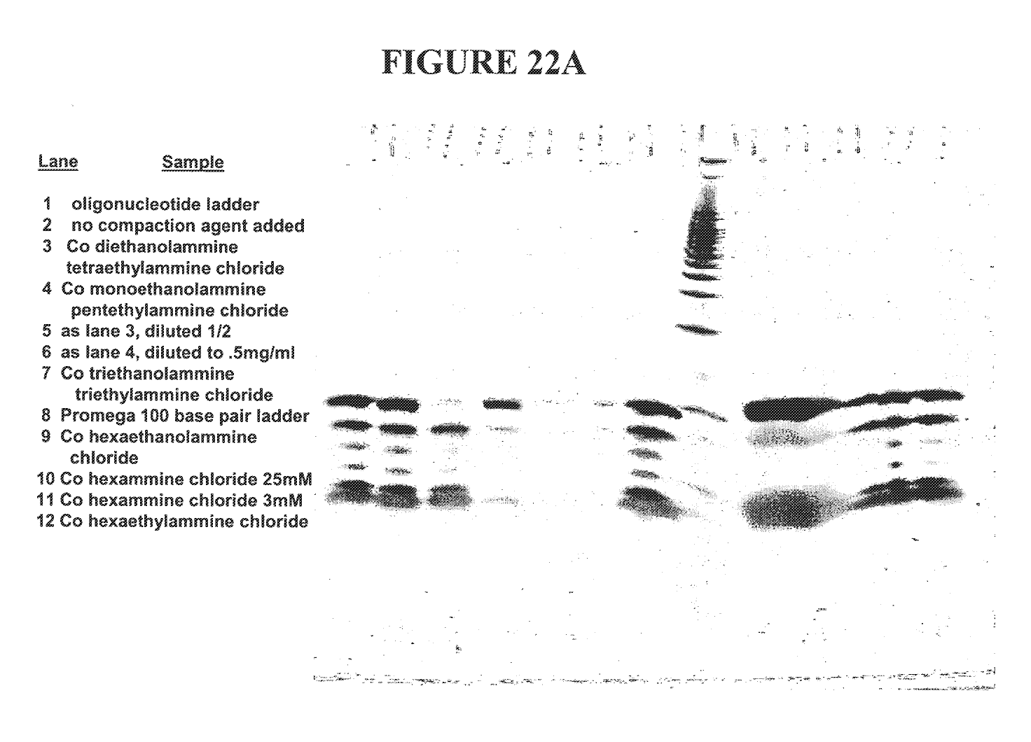

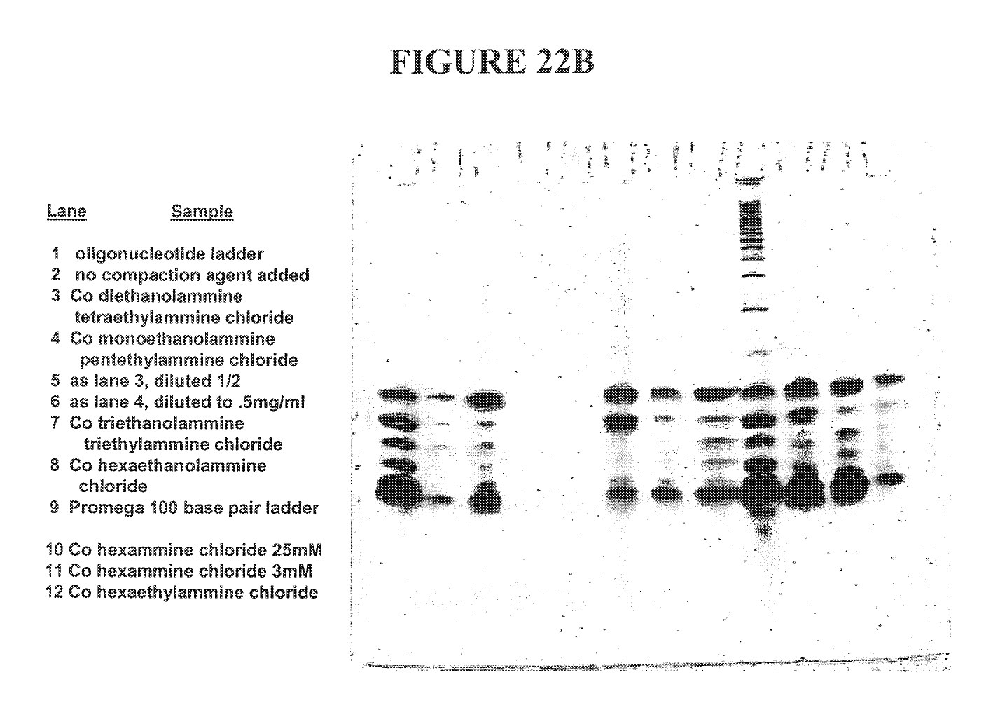

The samples were loaded onto a 15% acrylamide formamide gel, and separated by electrophoresis. The elution samples required about 2 hours at 60 volts in TBE buffer and the supernatant samples containing 9.0M acetamide/25 mM NaOAc, pH 5.2 required about 5 hours at 25 volts in TBE buffer (due to the significant salt effects on the separation). The results are shown in FIGS. 22A (supernatants) and 22B (elutions).

Lane 3 of FIG. 22A showed that the cobalt diethanolaminetetraethylamine chloride mixture bound DS DNA to the MagneSil particles, and lane 3 of FIG. 22B showed that the cobalt diethanolaminetetraethylamine chloride mixture eluted DS DNA from the MagneSil particles. Lane 3 of FIG. 22A showed relatively little binding of the other 5 oligonucleotide bands, and also little elution in lane 3 of FIG. 22B of the other 5 oligonucleotides. While the corresponding cobalt hexamine chloride samples in lanes 10 and 11 of FIG. 22A was obscured, lanes 10 and 11 of FIG. 22B showed that cobalt hexamine chloride promoted the elution of single stranded RNA from the MagneSil particles.

This examples demonstrates a simple method of screening transition metal complexes for their facilitation of binding of small RNA and DNA molecules to a binding matrix, and their elution therefrom. The relative absence of small DNA molecules in most samples allows for transition metal complexes with affinity for DNA to be useful in the present invention, although the concentration of transition metal complex used may need to be adjusted accordingly. Supplementation of the sample mixture with other compounds, such as alcohols, polyethylene glycol, salts such as NaCl, etc may provide a screening method suitable for other desired binding conditions (evaporation of alcohol prior to gel loading would likely be desirable).

### Example 30

### Quantitation of miR92 by qRT-PCR

In this example, small RNA purification was performed on CHO, HeLa, 3T3, and 293T cells followed by qRT-PCR of mature miR92 present in the eluate. 1×10⁶ of CHO, HeLa, 3T3, or 293T cultured cells were placed in each of 4 separate tubes in duplicate and washed twice with 200 μl 1×PBS pH 6.8 to remove cell culture media. PBS supernatant was removed after centrifugation of cells at 8,000×g for 5 minutes. To each of 4 tubes was added 200 μl of a mixture containing either 8M urea, 20 mM TRIS pH 7.5, 125 mM NaCl, and 25 mM Hexamminecobalt(III)chloride, or 9M acetamide, 115 mM TRIS pH 7.5, 125 mM NaCl, and 25 mM Hexamminecobalt(III)chloride. All tubes were vortexed and held at 21° C. for 5 minutes. 530 μl of 75% ethanol was added to each tube and vortexed. Each lysate was added to individual SV mini columns (Promega) and centrifuged at 2,000×g for 2 minutes. The flow through from each sample was discarded. All SV mini column membranes were washed twice with 500 μl of 75% ethanol (v/v with water) and centrifuged at 2,000×g for 1 minute. A final spin at 8,000×g for 5 minutes removed trace ethanol from the column membrane. 50 μl of nanopure water was added directly to the membrane of each column and held at 21° C. for 5 minutes. The eluate was captured in a fresh microfuge tube by spinning the column at 8,000×g for 2 minutes. A 5 μl portion of the each eluate was added to 10 μl of a prepared reverse transcription reaction and incubated following Applied Biosystem's (Foster City, Calif.) TAQMAN MicroRNA Reverse Transcription Kit (Part No.: 4366596) protocol. 20 μl Q-PCR reactions were prepared following Applied Biosystem's TAQMAN MicroRNA Assay human miR-21 kit (Part No.: 4373013) protocol. An 8 log standard curve was constructed by diluting a synthetic RNA oligo specific for the mature human miR92 to 0, 5, 502, 5017, 50167, 501667, 5016667, and 50166667 copies per reaction. Resulting data was analyzed using software on the Applied Biosystems 7500 qPCR platform and presented in Table 1.

TABLE 1

| | | miRNA 92 |
|---|---|---|
| Cell Type | Binding Soln | Copies per μl Eluate |
| CHO | Urea | 701163 |
| HeLa | Urea | 60467 |
| 3T3 | Urea | 172362 |
| 293T | Urea | 440750 |
| CHO | Acetamide | 492597 |
| HeLa | Acetamide | 958730 |
| 3T3 | Acetamide | 288126 |
| 293T | Acetamide | 355158 |

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A method for purifying small RNA molecules comprising:
a) preparing a sample comprising small RNA molecules, larger RNA molecules and compaction agent, wherein said small RNA molecules are less than 1000 bases in length, said longer RNA molecules are greater than 1000 bases in length, and said compaction agent comprises:
i) a plurality of metal-amine-halide molecules, wherein said metal-amine-halide molecules comprise a metal atom, a halide atom, and at least one amine group, or
ii) a plurality of metal-amine-salt molecules, wherein said metal-amine-salt molecules comprise a metal atom, a salt molecule, and at least one amine group;
b) contacting said sample with a single binding matrix such that a single RNA-bound binding matrix is generated, and
c) preferentially eluting small RNA molecules from said single RNA-bound binding matrix such that a purified small RNA preparation is generated, wherein said purified small RNA preparation comprises a plurality of eluted small RNA molecules, and wherein said purified small RNA preparation is substantially free of larger RNA molecules.

2. The method of claim 1, further comprising washing said RNA-bound binding matrix of step (b) with a wash solution.

3. The method of claim 1, wherein said sample in step a) further comprises DNA molecules, and wherein said purified small RNA preparation is substantially free of DNA molecules.

4. The method of claim 1, wherein said small RNA molecules are 500 bases in length or shorter.

5. The method of claim 1, wherein said small RNA molecules are 200 bases in length or shorter.

6. The method of claim 1, wherein said compaction agent comprises hexammine cobalt chloride.

7. The method of claim 1, wherein said sample further comprises a chaotropic agent, wherein said chaotropic agent comprises an amide.

8. The method of claim 7, wherein said chaotropic agent is selected from urea, thiourea, and acetamide.

9. The method of claim 1, wherein said sample further comprises a chaotropic agent, wherein said chaotropic agent comprises a urethane group.

10. The method of claim 1, wherein the concentration of said compaction agent in said sample prior to step b) is between about 2.0 mM and about 8.0 mM.

11. The method of claim 1, wherein said binding matrix comprises a membrane.

12. The method of claim 1, wherein said binding matrix comprises magnetic particles.

13. A method of reducing the degradation of RNA in a sample by RNase comprising contacting a sample comprising RNA and RNase with a compound selected from the group consisting of a chaotropic agent, a compaction agent and mixtures thereof.

14. A method for purifying small RNA molecules comprising:
a) providing a modified binding matrix comprising;
i) a compaction agent comprising:
A) a plurality of metal-amine-halide molecules, wherein said metal-amine-halide molecules comprise a metal atom, a halide atom, and at least one amine group, or
B) a plurality of metal-amine-salt molecules, wherein said metal-amine-salt molecules comprise a metal atom, a salt molecule, and at least one amine group; and
ii) a binding matrix, wherein at least a portion of said binding matrix is impregnated with, coated with, or impregnated and coated with said compaction agent;
b) contacting a sample with said modified binding matrix, wherein said sample comprises small RNA molecules and larger RNA molecules, and wherein said small RNA molecules are less than 1000 bases in length and said larger RNA molecules are greater than 1000 bases in length, such that an RNA-bound binding matrix is generated, and
c) preferentially eluting small RNA molecules from said RNA-bound binding matrix such that a purified small RNA preparation is generated, wherein said purified small RNA preparation comprises a plurality of eluted small RNA molecules, and wherein said purified small RNA preparation is substantially free of larger RNA molecules.

15. The method of claim 1, wherein after generation of the purified small RNA preparation, substantially all of the larger RNA molecules remain bound to the single binding matrix.

16. The method of claim 1, wherein the compaction agent is selected from the group consisting of nickel hexammine chloride, ruthenium hexamine trichloride, nickel hexamethylammine chloride, nickel hexaethylammine chloride, cobalt hexamethylammine chloride, cobalt hexaethylammine chloride.

17. A method for purifying small RNA molecules comprising:
a) preparing a sample comprising small RNA molecules, larger RNA molecules and a compaction agent, the small RNA molecules being less than 1000 bases in length, the larger RNA molecules being greater than 1000 bases in length, and the compaction agent comprising a metal-amine-halide or a metal-amine-salt;
b) contacting the sample with a single binding matrix to generate an RNA-bound binding matrix; and
c) preferentially eluting small RNA molecules from the RNA-bound binding matrix to generate a purified small RNA preparation, wherein the purified small RNA preparation comprises small RNA molecules, and is substantially free of larger RNA molecules.

18. The method of claim 17, wherein the sample is prepared by contacting the compaction agent with a lysate, and the method does not comprise a separate lysate purification step.

19. The method of claim 17, wherein the binding matrix is a single binding column membrane.

20. The method of claim 17, wherein after generation of the purified small RNA preparation, substantially all of the larger RNA molecules remain bound to the single binding matrix.

21. The method of claim 17, wherein the binding matrix is a magnetic silica particle.

22. The method of claim 17, further comprising washing the RNA-bound binding matrix with a wash solution.

23. The method of claim 17, wherein the sample comprises DNA, and the purified small RNA preparation is substantially free of DNA.

24. The method of claim 17, wherein said compaction agent comprises hexammine cobalt chloride.

25. The method of claim 17, wherein said sample further comprises a chaotropic agent, wherein said chaotropic agent comprises an amide.

26. The method of claim 25, wherein the chaotropic agent comprising an amide is urea, thiourea, or acetamide.

27. The method of claim 17, wherein said sample further comprises a chaotropic agent, wherein said chaotropic agent comprises a urethane group.


SEQUENCE LISTING (6 sequences)

SEQ ID NO: 1 - RNA, 21 nt, Artificial Sequence
  Synthetic
uauugcacuugucccggccug

SEQ ID NO: 2 - RNA, 25 nt, Artificial Sequence
  Synthetic
gagacccaguagccagauguagcuu

SEQ ID NO: 3 - RNA, 25 nt, Artificial Sequence
  Synthetic
aagcuacaucuggcuacugggucuc

SEQ ID NO: 4 - DNA, 35 bp, Artificial Sequence
  Synthetic
agctgtctaggtgacacgctagagtactcgagcta

SEQ ID NO: 5 - DNA, 35 bp, Artificial Sequence
  Synthetic
tagctcgagtactctagcgtgtcacctagacagct

SEQ ID NO: 6 - DNA, 30 bp, Artificial Sequence
  Synthetic
gttacacatgcctacacgctccatcatagg